(19) United States
(12) Patent Application Publication
(10) Pub. No.: US 2012/0077758 A1
(43) Pub. Date: Mar. 29, 2012

(54) INFERTILITY ASSOCIATED DEFB-126 DELETION POLYMORPHISM

(75) Inventors: Charles L. Bevins, Davis, CA (US); Tsang Lau, New York, NY (US); Gary N. Cherr, Penngrove, CA (US); Ashley I. Yudin, Davis, CA (US); Theodore L. Tollner, Wilton, CA (US); James W. Overstreet, Davis, CA (US); Scott Venners, Burnaby (CA); Xiping Xu, Wilmette, IL (US)

(73) Assignees: Board of Trustees of the University of Illinois, Urbana, IL (US); The Regents of the University of California, Oakland, CA (US)

(21) Appl. No.: 13/203,467

(22) PCT Filed: Feb. 26, 2010
(86) PCT No.: PCT/US2010/025626

§ 371 Date: Nov. 22, 2011

Related U.S. Application Data
(60) Provisional application No. 61/155,807, filed on Feb. 26, 2009.

(51) Int. Cl. A61K 38/17; G01N 33/53; C12N 5/076; A61P 15/08; C12Q 1/68; G01N 33/566

(52) U.S. Cl. 514/21.2; 435/6.11; 436/501; 435/375

(57) ABSTRACT

The present application provides diagnostic methods for determining the fertility status of a male individual by evaluating his DEFB-126 phenotypic and genotypic status. The present invention relates to a dinucleotide deletion polymorphism in the protein coding sequence of a DEFB-126 nucleic acid. The amino acid sequence of this variant has a significantly altered the carboxyl terminal, carbohydrate-containing domain of DEFB-126 in comparison to a wild-type DEFB-126 polypeptide. This variant results in aberrant protein function and structure, leading to reduced sperm function and fertility. The present invention provides methods for analyzing the genotype of individuals with respect to the gene encoding DEFB-126 in order to determine whether that individual has reduced fertility. Such determination will provide an individual knowledge of whether their genotype is associated with a risk of reduced fertility and to allow that individual to receive appropriate fertility treatment options. The present invention further provides kits that are useful for diagnosing increased risk or probability of infertility based on the presence or absence of the DEFB-126 deletion polymorphism. The application also provides therapeutic methods and compositions for restoring sperm functionality (e.g., to effect conception) in sperm from an individual who expresses insufficient levels of DEFB-126.

### CROSS REFERENCE TO RELATED APPLICATIONS

This application claims the benefit of U.S. Provisional Application No. 61/155,807, filed on Feb. 26, 2009, the entire disclosure of which is hereby incorporated herein by reference.

### STATEMENT AS TO RIGHTS TO INVENTIONS MADE UNDER FEDERALLY SPONSORED RESEARCH AND DEVELOPMENT

The U.S. Government has a paid-up license in this invention and the right in limited circumstances to require the patent owner to license to others on reasonable terms as provided for by the terms of Grant Nos. AI032738 and AI050843, awarded by the National Institutes of Health.

### FIELD OF THE INVENTION

The present application provides diagnostic methods for determining the fertility status of a male individual by evaluating his DEFB-126 phenotypic and genotypic status. The application also provides therapeutic methods and compositions for restoring sperm functionality (e.g., to effect conception) in sperm from an individual who expresses insufficient levels of DEFB-126. In some embodiments, the invention provides methods and compositions for using polymorphisms in the DEFB-126 gene to determine whether an individual has an increased risk or probability of infertility.

### BACKGROUND OF THE INVENTION

Human infertility is generally defined as the inability to achieve pregnancy after one year of sexual intercourse without contraception. By this definition, the prevalence of infertility in many countries of the world is approximately 13-14% (Strickler et al., Am. J. Obstet. Gynecol. 172:766-73 (1995)). Infertility in males is usually assessed by analysis of parameters of semen quality including sperm concentration in the ejaculate, the percentage of motile sperm and the percentage of sperm with normal morphology, but none of these measures are diagnostic of infertility (Guzick et al., N. Engl. J. Med. 345:1388-93 (2001)). The prevalence of unexplained infertility has been estimated to be approximately 17% of infertile couples (Collins, Unexplained Infertility. In: Infertility Evaluation and Treatment (Keye, Chang, Rebar, and Soules, eds.) W B Saunders, Philadelphia, pages 249-262 (1995)). In these cases, no abnormalities of reproductive function can be established in either the male or female partner.

There exists a need to diagnose unexplained infertility. The present invention fulfills these and other needs, as will be apparent upon review of the following disclosure.

### BRIEF SUMMARY OF THE INVENTION

Knowledge of the causes of infertility allows for rapid progression to directed interventions for couples seeking to achieve pregnancy. The present invention is based, in part, on the discovery that a two nucleotide deletion in the gene that codes for DEFB-126 (herein “DEFB-126 deletion polymorphism”) significantly increases the probability that the individual possessing the polymorphism will be infertile.

In one aspect, the present invention provides compositions and methods for evaluating the presence or absence of a DEFB-126 deletion polymorphism to determine an individual's risk or likelihood for increased infertility. In one aspect, the invention provides a method for determining whether an individual has an increased probability of infertility comprising: determining the DEFB-126 alleles of the individual within the subsequence TCCTACCCCCGTTTC (SEQ ID NO:1) of a nucleic acid encoding DEFB-126, wherein the presence of five contiguous cytosines “CCCCC” at positions 6-10 within the subsequence is indicative of normal fertility and the presence of at most three contiguous cytosines “CCC” at positions 6-10 of the subsequence is indicative of an increased risk or likelihood of infertility.

In some embodiments, the methods comprise determining the DEFB-126 alleles within the subsequence ATGGCTCCTACCCCCGTTTCTCCCA (SEQ ID NO:2) of a nucleic acid encoding DEFB-126, wherein the presence of five contiguous cytosines “CCCCC” at positions 11-15 within the subsequence is indicative of normal fertility and the presence of at most three contiguous cytosines “CCC” at positions 11-15 of the subsequence is indicative of an increased risk or probability of infertility.

In some embodiments, the individuals is human. In some embodiments, the individual is male.

In some embodiments, the nucleic acid is DNA, and in other embodiments, the nucleic acid is RNA.

In some embodiments, the nucleic acid encoding DEFB-126 shares at least 95%, 96%, 97%, 98%, 99% sequence identity to SEQ ID NO:4. In some embodiments, the nucleic acid encoding DEFB-126 shares at least 95%, 96%, 97%, 98%, 99% sequence identity to a nucleic acid selected from SEQ ID NO:5, SEQ ID NO:13 and SEQ ID NO:14.

The DEFB-126 deletion polymorphism can be detected by any method known in the art. In some embodiments, the DEFB-126 deletion polymorphism is detected by an amplification reaction. With respect to detecting a polymorphism using amplification reactions, the DEFB-126 alleles can be detected by an amplification reaction using one or more polynucleotides that distinguish between alleles within the subsequence TCCTACCCCCGTTTC (SEQ ID NO:1) of a nucleic acid encoding DEFB-126. In some embodiments, the amplification reaction is selected from the group consisting of polymerase chain reaction (PCR), strand displacement amplification (SDA), nucleic acid sequence based amplification (NASBA), rolling circle amplification (RCA), T7 polymerase mediated amplification, T3 polymerase mediated amplification, and SP6 polymerase mediated amplification.

In some embodiments, the DEFB-126 alleles are detected by hybridization using one or more polynucleotides that distinguish between alleles within the subsequence TCCTACCCCCGTTTC (SEQ ID NO:1) of a nucleic acid encoding DEFB-126. In other embodiments, the DEFB-126 alleles are detected by sequencing a subsequence of DEFB-126, the subsequence comprising the nucleic acid sequence TCCTACCCCCGTTTC (SEQ ID NO:1). In some embodiments, the DEFB-126 alleles are detected by restriction fragment length polymorphism. In other embodiments, the DEFB-126 alleles are detected by fluorescence resonance energy transfer (“FRET”).

Other aspects of the present invention analyze variants of the DEFB-126 polypeptide to determine an individual's risk or probability of infertility. One embodiment determines whether an individual has an increased risk of infertility comprising obtaining a biological sample from the individual and determining the presence of a DEFB-126 polypeptide in the sample, wherein the presence of a DEFB-126 polypeptide is indicative of normal fertility, and the absence (or reduced presence) of a DEFB-126 polypeptide is indicative of an increased probability of infertility.

In some embodiments, the DEFB-126 polypeptide indicative of normal fertility shares at least 95%, 96%, 97%, 98%, 99% sequence identity to a wild-type DEFB-126 polypeptide selected from SEQ ID NO:6, SEQ ID NO: 7, SEQ ID NO:8, SEQ ID NO:9, SEQ ID NO:10, SEQ ID NO:11 and SEQ ID NO:12. In some embodiments, the DEFB-126 polypeptide indicative of normal fertility shares at least 95%, 96%, 97%, 98%, 99% sequence identity to a wild-type DEFB-126 polypeptide of SEQ ID NO:6. In some embodiments, the DEFB-126 polypeptide indicative of normal fertility shares at least 95%, 96%, 97%, 98%, 99% sequence identity to a wild-type DEFB-126 polypeptide of SEQ ID NO:12.

In some embodiments, the variant DEFB-126 polypeptide indicative of an increased risk of infertility shares at least 95%, 96%, 97%, 98%, 99% sequence identity to SEQ ID NO:16, a variant DEFB-126 polypeptide expressed from a DEFB-126 nucleic acid having the dinucleotide deletion.

In some embodiments, a DEFB-126 polypeptide having a C-terminal amino acid sequence of TVSPTG (SEQ ID NO:35) is indicative of normal fertility. In some embodiments, a DEFB-126 polypeptide having a C-terminal amino acid sequence of RFSHWLNIPASVSCSRIPDSLKQRGL(K)ₙ (SEQ ID NO:18) is indicative of an increased risk of infertility.

In an aspect of the invention, an antibody is used as a method to determine infertility. In some embodiments, the DEFB-126 polypeptide is determined using an antibody that binds specifically to the C-terminus of the polypeptide. The antibody can bind specifically to the C-terminus of either a wild-type or variant DEFB-126 polypeptide. In some embodiments, the variant DEFB-126 polypeptide is determined by ELISA, immunoprecipitation, immunoaffinity chromatography, protein array, lectin binding, isoelectric focusing or Western blot.

Another aspect of the invention provides a method for determining whether an individual has an increased risk of infertility comprising obtaining a sperm sample from the individual and contacting the sample with a lectin that selectively binds Galactose-GalNAc or sialic acid, wherein the absence of or a reduced binding level of the lectin in comparison to a normal control or a predetermined threshold level is indicative of an increased risk of infertility. In some embodiments, the lectin is Agaricus bisporus (ABA) or Artocarpus integrifolia (Jacalin). Significant reduction in sialic acid moieties on the sperm surface has been demonstrated with loss of DEFB126 from non-human primate sperm. Accordingly, reduced binding levels of lectins which recognize sialic acid moieties in terminal positions on oligosaccharides on DEFB126 in comparison to a normal control or a predetermined threshold level is therefore indicative of increased risk of infertility. In some embodiments, the lectin is Limulus polphemus (LPA), Macackia amurenesis (MAL II), or Triticum vulgaris (WGA).

Another aspect of the invention provides a method for determining whether an individual has an increased risk of infertility comprising obtaining a sperm sample from the individual and contacting the sample with poly-L-lysine, (or other poly cationic substance) which binds to sialic acid (and other negatively charged glycan residues on the sperm surface), wherein the absence of or a reduced binding level of the poly-L-lysine (or poly cationic substance) in comparison to a normal control or a predetermined threshold level is indicative of an increased risk of infertility. The loss of DEFB 126 from the surface of non-human primate is associated with significant reductions in sperm binding to poly-L-lysine (or other poly cationic substance). Accordingly, reduced binding levels of poly-L-lysine which recognize negatively charged moieties associated with oligosaccharides on DEFB126 in comparison to a normal control or predetermined threshold level is therefore indicative of increased risk of infertility.

In some embodiments, the methods further comprise determining and/or selecting an appropriate treatment for infertility. In some embodiments, the methods further comprise selecting appropriate diagnostic tests to identify the reason for infertility. In some embodiments, the methods further comprise recording on a tangible medium, e.g., on paper or in an electronic or computer file, the results of the determination of the presence or absence of a DEFB-126 deletion polymorphism.

The present invention further provides kits. In one embodiment, a kit for determining whether an individual has an increased risk of infertility is provided, the kit comprising at least one polynucleotide that distinguishes the DEFB-126 alleles of the individual within the subsequence TCCTACCCCCGTTTC (SEQ ID NO:1), and instructions indicating that the presence of five contiguous cytosines “CCCCC” at positions 6-10 within the subsequence is indicative of normal fertility and the presence of three contiguous cytosines “CCC” at positions 6-10 of the subsequence is indicative of an increased risk of infertility.

In other embodiments, a kit for determining whether an individual has an increased risk of infertility is provided, the kit comprising at least one antibody that recognizes a DEFB-126 polypeptide, and instructions indicating that the presence of a DEFB-126 polypeptide is indicative of normal fertility, and the absence (or reduced presence) of a DEFB-126 polypeptide is indicative of an increased probability of infertility. In some embodiments, the kits contain at least one antibody that binds specifically to the C-terminus of a DEFB-126 polypeptide. The antibody can bind specifically to the C-terminus of either a wild-type or variant DEFB-126 polypeptide.

In other embodiments, a kit for determining whether an individual has an increased risk of infertility is provided, the kit comprising at least one lectin that recognizes a DEFB-126 polypeptide, and instructions indicating that the presence of a DEFB-126 polypeptide (demonstrated by binding of the lectin) is indicative of normal fertility, and the absence (or reduced presence) of a DEFB-126 polypeptide is indicative of an increased probability of infertility. In some embodiments, the kits contain at least one lectin that selectively binds Galactose-GalNAc or sialic acid. In some embodiments, the lectin is Agaricus bisporus (ABA) or Artocarpus integrifolia (Jacalin). In some embodiments, the lectin is Limulus polphemus (LPA), Macackia amurenesis (MAL II), or Triticum vulgaris (WGA). In some embodiments, the lectin comprises a detectable label, for example, a fluorophore, an enzyme, a chemiluminescent moiety, a chromophore, etc.

In other embodiments, a kit for determining whether an individual has an increased risk of infertility is provided, the kit comprising poly-L-lysine (or similar polycationic substance) that recognizes negatively charged moieties associated with a DEFB-126 polypeptide, and instructions indicating that the presence of a DEFB-126 polypeptide (demonstrated by binding of the poly-L-lysine or polycation) is indicative of normal fertility, and the absence (or reduced presence) of a DEFB-126 polypeptide is indicative of an increased probability of infertility. In some embodiments, poly-L-lysine comprises a detectable label, for example, a fluorophore, an enzyme, a chemiluminescent moiety, a chromophore, etc.

The present invention further provides a method for treating a male individual with reduced fertility resulting from a nonfunctional variant DEFB-126 polypeptide comprising introducing into an epididymis cell from the individual a nucleic acid encoding a functional DEFB-126 polypeptide.

The invention further provides methods for restoring or improving sperm functionality in sperm from an individual who expresses insufficient levels of functional

DEFB-126 to effect conception, comprising contacting a sperm sample obtained from the individual with a functional DEFB-126 polypeptide. In some embodiments, the individual is a human and the functional DEFB-126 polypeptide is a human DEFB-126 polypeptide. In some embodiments, the individual is a human and the functional DEFB-126 polypeptide is a non-human DEFB-126 polypeptide or a DEFB-126 polypeptide mimetic. In some embodiments, the individual is a human and the functional DEFB-126 polypeptide is a non-human primate DEFB-126 polypeptide.

In some embodiments, the sperm is contacted in vitro with a functional DEFB-126 polypeptide. In some embodiments, the sperm is contacted intravaginally with a functional DEFB-126 polypeptide.

The DEFB-126 polypeptide or peptide mimetic useful in the present compositions is one that is capable of allowing for normal fertility. A functional DEFB-126 polypeptide has two general properties of the native DEFB126 molecule: (1) the ability to bind reversibly to the sperm surface depending on sperm capacitation state, and (2) the ability to impart a negative charge to the sperm surface while bound.

Accordingly, in some embodiments, the functional DEFB-126 polypeptide or polypeptide mimetic comprises a core beta-defensin motif (aa 21-67), e.g., a polypeptide comprising an amino acid sequence having 95%, 96%, 97%, 98%, 99% or 100% sequence identity to SEQ ID NO:46, SEQ ID NO:47, SEQ ID NO:48, SEQ ID NO:48 or SEQ ID NO:49. Where there are no shared residues among the orthologs (−) (aligned above), amino acids can be substituted that are similar in charge or polarity or that contribute to retention of charge and polarity of inter-cysteine spans.

In some embodiments, the DEFB-126 polypeptide or polypeptide mimetic comprises a carboxyl extension motif (e.g., aa 68-121, 68-134; or 68-181) that is sufficiently anionic to impart a negative charge to the sperm surface while bound, e.g., has a sufficient number of N-linked carbohydrates, e.g., sialic acid moieties.

In some embodiments, the functional DEFB-126 polypeptide or polypeptide mimetic comprises a defensin core motif and a defensin carboxyl extension motif. In some embodiments, the functional DEFB-126 polypeptide or polypeptide mimetic comprises a defensin core motif and a carboxy motif that comprises one or more tandem repeats or sequence segments that allow for O-linked and/or N-linked glycosylation (e.g., mucin repeat sequences) such that the polypeptide is sufficiently anionic to impart a negative charge to the sperm surface while bound. In some embodiments, the functional DEFB-126 polypeptide or polypeptide mimetic comprises a defensin core motif of SEQ ID NOs: 46, 47, 48 or 49 and a defensin carboxyl extension motif of SEQ ID NO:50, or shorter lengths of SEQ ID NO:50 (e.g., aa 68-121, 68-134; or 68-181) with sufficient anionic charge to impart a negative charge to the sperm surface while bound.

In some embodiments of the reconstitutional methods, the functional DEFB-126 polypeptide comprises an amino acid sequence that is at least 95% identical to a sequence selected from the group consisting of SEQ ID NO:6, SEQ ID NO: 7, SEQ ID NO:8, SEQ ID NO:9, SEQ ID NO:10, SEQ ID NO:11 and SEQ ID NO:12. In some embodiments, the functional DEFB-126 polypeptide comprises an amino acid sequence that is at least 95% identical to a sequence selected from the group consisting of SEQ ID NO:6 and SEQ ID NO:12. In some embodiments, the functional DEFB-126 polypeptide comprises an amino acid sequence that is at least 95% identical to SEQ ID NO:6. In some embodiments, the functional DEFB-126 polypeptide comprises SEQ ID NO:6. In some embodiments, the functional DEFB-126 polypeptide comprises an amino acid sequence that is at least 95% identical to SEQ ID NO:12. In some embodiments, the functional DEFB-126 polypeptide comprises SEQ ID NO:12.

The invention further provides compositions comprising a functional DEFB-126 polypeptide and a pharmaceutically acceptable carrier. In some embodiments, the functional DEFB-126 polypeptide is a human DEFB-126 polypeptide. In some embodiments, the functional DEFB-126 polypeptide is a non-human DEFB-126 polypeptide or a DEFB-126 polypeptide mimetic. In some embodiments, the functional DEFB-126 polypeptide is a non-human primate DEFB-126 polypeptide.

Further embodiments of the functional DEFB-126 polypeptide or polypeptide mimetic for use in the compositions are as described above and herein.

In some embodiments, the composition is a foam, for example, a foam formulated for intravaginal delivery.

In a related embodiment, the invention provides methods for first diagnosing whether an individual is deficient for functional DEFB-126, e.g., by either a phenotypic or genotypic analysis, as described herein, and if the individual is determined to be DEFB-126 deficient, then contacting the sperm of that individual with a functional DEFB-126 polypeptide, as described herein.

### DEFINITIONS

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry and nucleic acid chemistry and hybridization described below are those well known and commonly employed in the art. Standard techniques are used for nucleic acid and peptide synthesis. Generally, enzymatic reactions and purification steps are performed according to the manufacturer's specifications. The techniques and procedures are generally performed according to conventional methods in the art and various general references (see generally, Sambrook et al. Molecular Cloning: A Laboratory Manual, 3rd ed., Cold Spring Harbor Laboratory Press, Cold Spring Harbor, N.Y. (2001) and Ausubel, ed., Current Protocols in Molecular Biology, John Wiley Interscience, (1990-2008)), which are provided throughout this document. The nomenclature used herein and the laboratory procedures in analytical chemistry, and organic synthetic described below are those well known and commonly employed in the art. Standard techniques, or modifications thereof, are used for chemical syntheses and chemical analyses.

Biological samples refer to the solid tissue or a biological fluid that contains either a DEFB-126 nucleic acid or expressed protein, with or without the deletion polymorphism. With respect to nucleic acids, the biological sample can be tested by the methods described herein and include body fluids including whole blood, serum, plasma, cerebrospinal fluid, urine, lymph fluids, semen, sperm cells, and various external secretions of the respiratory, intestinal and genitourinary tracts, tears, saliva, milk, white blood cells, myelomas, and the like; and biological fluids such as cell extracts, cell culture supernatants; fixed tissue specimens; and fixed cell specimens. Biological samples can also be from solid tissue, including hair bulb, skin, biopsy or autopsy samples or frozen sections taken for histologic purposes. These samples are well known in the art. A biological sample is obtained from any individual to be tested for the DEFB-126 deletion polymorphism. In some embodiments, the biological sample is semen or sperm cells. A biological sample can be suspended or dissolved in liquid materials such as buffers, extractants, solvents and the like.

Normal fertility refers to an approximately 80-85% chance of becoming pregnant within 21 months for couples attempting to conceive, as the prevalence of infertility in many countries in the world is approximately 13-14% (see Strickler et al., Am. J. Obstet. Gynecol. 172:766-73 (1995)).

Human infertility is generally defined as the inability to achieve pregnancy after one year of sexual intercourse without contraception.

As used herein, an “increased risk or probability or likelihood of infertility” or “reduced fertility” interchangeably refer to a reduction of odds to under 70% chance of becoming pregnant within 21 months for couples attempting to conceive. In some embodiments, this can be compared against a population with normal fertility.

A gene refers to a hereditary unit consisting of a sequence of DNA that has a specific chromosomal location. A gene is expressed to produce a protein product.

An allele refers to a particular variation of a gene. As it pertains to this invention, an allele may be either a wild-type copy of DEFB-126, or the DEFB-126 deletion polymorphism, as described herein.

The terms “nucleic acid” and “polynucleotide” are used interchangeably herein to refer to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

Structurally, a DEFB-126 or wild-type DEFB-126 refers to nucleic acids and polypeptide polymorphic variants, alleles, mutants, and interspecies homologs that: (1) have an amino acid sequence that has greater than about 90% amino acid sequence identity, for example, 91%, 92%, 93%, 94%, 95%, 96%, 97%, 98% or 99% or greater amino acid sequence identity, preferably over a region of at least about 25, 50, 100, 200, 500, 1000, or more amino acids, or over the full-length, to an amino acid sequence encoded by a DEFB-126 nucleic acid (see, e.g., SEQ ID NO:4 and SEQ ID NO:5 (human), and GenBank Accession No. NM_{—}030931 (human)) or to an amino acid sequence of a DEFB-126 polypeptide (see e.g. SEQ ID NO:6 (human), SEQ ID NO:7 (Hylobates lar), SEQ ID NO:8 (Gorilla), SEQ ID NO:9 (Pantrolgoldytes), SEQ ID NO:10 (Macaca fascicularis), SEQ ID NO:11 (Pongo pygmaeus), GenBank Accession Nos. NP_{—}112193.1 (human), A4H245.1 (Hylobates lar), A4H243.1 (Gorilla), XP_{—}514453 (Pan troglodytes) CAL68961.1 (Macaca fascicularis) and A4H244.1 (Pongo pygmaeus)); (2) bind to antibodies, e.g., polyclonal antibodies, raised against an immunogen comprising an amino acid sequence of a DEFB-126 polypeptide (e.g., encoded by a nucleic acid sequence of SEQ ID NO:4, SEQ ID NO:5 or a nucleic acid of GenBank Accession No NM_{—}030931); or an amino acid sequence (e.g., encoded by SEQ ID NO:6, SEQ ID NO:7, SEQ ID NO:8, SEQ ID NO:9, SEQ ID NO:10, SEQ ID NO:11, or GenBank Accessions Nos. NP_{—}112193.1, A4H245.1, A4H243.1, XP_{—}514453, CAL68961.1, A4H244.1), and conservatively modified variants thereof; (3) specifically hybridize under stringent hybridization conditions to an anti-sense strand corresponding to a nucleic acid sequence encoding a DEFB-126 protein, and conservatively modified variants thereof; (4) have a nucleic acid sequence that has greater than about 95%, preferably greater than about 96%, 97%, 98%, 99%, or higher nucleotide sequence identity, preferably over a region of at least about 25, 50, 100, 200, 500, 1000, or more nucleotides, or over the full-length, to a DEFB-126 nucleic acid (e.g., SEQ ID NO:4, SEQ ID NO:5). Wild-type DEFB-126 alleles have five contiguous cytosines “CCCCC” within positions 6-10 of the contextual subsequence TCCTACCCCCGTTTC of SEQ ID NO:1.

All defensins have a largely β-sheet structure and contain 3 intramolecular cysteine disulfide bonds. β-defensins are defined by a six-cysteine motif with usual spacing of C—X₆—C—X₃₋₄—C—X₉₋₁₂—C—X₅₋₆-C—C (SEQ ID NO:19) and a large number of basic amino acid residues. Likewise, DEFB-126 is a peptide with the canonical core of cysteine residues, and a C-terminal tail of 52 amino acids, yielding a molecular weight of about 12,000 Da (based on deduced amino acid sequence). Amino acid sequence analysis of wild-type human DEFB-126 identifies at least 20 sites for O-linked glycosylation within the C-terminal tail. Primate DEFB-126 polypeptides share a high level of sequence identity (see, FIG. 12; Perry, et al., Biol. Reprod. 61:965-972 (1999); Schutte, et al., PNAS 99(4):2129-2133 (2002); and Rodriguez-Jiménez, Genomics 81:175-183 (2003)).

Following is an alignment of the defensin core region (aa 21-67) from human (SEQ ID NO:46), macaque (SEQ ID NO:47) and mouse (SEQ ID NO:48), as well as a consensus sequence (“con”) (SEQ ID NO:49). Conserved cyteines are blocked; conserved residues are shaded.

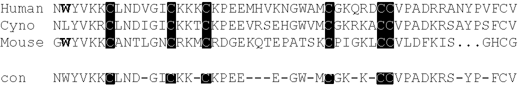

Those of skill recognize that amino acid residues conserved between species and different β-defensin proteins generally are less tolerant of substitution or deletion. For example, the cysteine residues contributing to the disulfide-stabilized core of a DEFB-126 protein should not be substituted or deleted. Conversely, amino acid residues not conserved between species and different β-defensin proteins can oftentimes be substituted or deleted without affecting the function of the protein.

Functionally, wild-type DEFB-126 operates in the capacitation of primate (human and non-human) spermatozoa and modulates sperm surface-receptor presentation at the time of fertilization (Tollner et al., Mol. Reprod. Dev. 69:327-37 (2004)). DEFB 126 also protects the entire primate sperm surface from immune recognition and the sialic acid moieties are responsible for the cloaking characteristic of this unique glycoprotein (Yudin et al., Biol. Reprod. 73:1243-1252 (2005)). The sialic acid moieties of DEFB-126 oligosaccharides are also responsible for facilitating the movement of sperm through cervical mucus (Tollner et al., Human Reprod. 23:2523-34 (2008)). DEFB-126 further mediates attachment of non-human primate sperm to oviductal epithelia, potentially a mechanism involved in the formation of an oviductal reservoir (Tollner et al., Biol. Reprod. 78:400-412 (2008)).

As used herein, a DEFB-126 deletion polymorphism refers to a two-nucleotide “CC” deletion within the contextual subsequence of a wild-type DEFB-126 nucleotide sequence defined by TCCTACCCCCGTTTC (SEQ ID NO:1). A nucleic acid encoding a DEFB-126 polypeptide with the deletion polymorphism will not contain more than three cytosines within positions 6-10 of the contextual subsequence TCCTACCCCCGTTTC of SEQ ID NO:1. See also FIG. 2. Exemplary DEFB-126 nucleic acid sequences with a DEFB-126 deletion polymorphism include SEQ ID NO:13 (GenBank Accession No. AK225987), SEQ ID NO:14, and SEQ ID NO:15 (GenBank Accession No. CO408416). The DEFB-126 deletion polymorphism frame shift causes a “read through” of the native stop codon, thereby producing a DEFB-126 variant polypeptide with an extended C-terminal region (e.g., SEQ ID NOS:16-18 and FIGS. 2 and 3) in comparison to the native protein (e.g., SEQ ID NOS:3, 6-12 and FIG. 1).

A variant DEFB-126 polypeptide refers to the resultant protein expressed from a nucleic acid sequence having the DEFB-126 deletion polymorphism. The protein product of the DEFB-126 deletion polymorphism, when expressed, contains an extended C-terminus (see e.g., SEQ ID NOs:16-18) in comparison to the wild-type DEFB-126 C-terminal region (e.g., SEQ ID NOs:3, 6-12). See also, FIG. 3. An extended C-terminus refers to an elongated portion of the C-terminal domain that begins at the motif SMS(S/L)M(A/T) (SEQ ID NO:20), e.g., at amino acid 106 of SEQ ID NO:16. This extended C-terminus causes a profound alteration in the structure and function of the DEFB-126 protein, most notably a lack of oligosaccharides in the region immediately C-terminal to the amino acid sequence SMS(S/L)M(A/T) (SEQ ID NO:20) of the wild-type DEFB-126 polypeptide sequence (e.g., SEQ ID NOS:6-12).

A nucleic acid “that distinguishes” as used herein refers to a polynucleotide(s) that (1) specifically hybridizes under stringent hybridization conditions to an anti-sense strand corresponding to a nucleic acid sequence encoding a DEFB-126 protein, and conservatively modified variants thereof; or (2) has a nucleic acid sequence that has greater than about 80%, 85%, 90%, 95%, preferably greater than about 96%, 97%, 98%, 99%, or higher nucleotide sequence identity, preferably over a region of at least about 20, 25, 50, 100, 200, 500, 1000, or over the full length to a DEFB-126 nucleic acid (e.g., a sequence as set forth in SEQ ID NO:4 and SEQ ID NO:5), or complements, subsequences, or consensus sequences between human and primates (see e.g., FIG. 12) thereof. A nucleic acid that distinguishes a DEFB-126 deletion polymorphism from a wild-type DEFB-126 nucleic acid sequence that does not contain a deletion polymorphism will allow for polynucleotide extension and amplification after annealing to a DEFB-126 polynucleotide comprising the deletion polymorphism, but will not allow for polynucleotide extension or amplification after annealing to a DEFB-126 polynucleotide that does not contain the deletion polymorphism. In other embodiments, a nucleic acid that distinguishes a DEFB-126 deletion polymorphism from a DEFB-126 nucleic acid sequence that does not contain a deletion polymorphism will hybridize to a DEFB-126 polynucleotide comprising the deletion polymorphism but will not hybridize to a DEFB-126 polynucleotide that does not contain the deletion polymorphism.

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology--Hybridization with Nucleic Probes, “Overview of principles of hybridization and the strategy of nucleic acid assays” (1993). Generally, stringent conditions are selected to be about 5-10° C. lower than the thermal melting point I for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, optionally 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein that is the predominant species present in a preparation is substantially purified. In particular, an isolated DEFB-126 nucleic acid is separated from open reading frames that flank the DEFB-126 gene and encode proteins other than DEFB-126. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

A polypeptide variant refers to a polypeptide that is produced as a result of expression from a gene sequence that is not wild-type. As it is referred to in herein, the variant DEFB-126 polypeptide is the protein produced as a result of the DEFB-126 deletion polymorphism. The variant DEFB-126 polypeptide contains an extended C-terminal domain relative to the polypeptide produced by expression of wild-type DEFB-126.

A “functional DEFB-126 polypeptide” refers to a DEFB-126 polypeptide that can be adsorbed to the surface of a sperm cell, e.g., that facilitates sperm capacitation. A functional DEFB-126 polypeptide can contain at least 20 sites for O-linked glycosylation.

A “nonfunctional DEFB-126 polypeptide” refers to a DEFB-126 polypeptide is not adsorbed to the surface of a sperm cell, e.g., that does not facilitate sperm capacitation. A nonfunctional DEFB-126 polypeptide may contain a significant reduction in O-linked glycosylation sites.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, α-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:

1) Alanine (A), Glycine (G);

2) Aspartic acid (D), Glutamic acid (E);

3) Asparagine (N), Glutamine (Q);

4) Arginine I, Lysine (K);

5) Isoleucine (I), Leucine (L), Methionine (M), Valine (V);

6) Phenylalanine (F), Tyrosine (Y), Tryptophan (W); and

7) Serine (S), Threonine (T) (see, e.g., Creighton, Proteins (1984)).

An antibody refers to either a polyclonal or monoclonal antibody that is able to recognize a specified protein. Antibodies may be generated that are able to recognize either an entire protein, or short peptide sequences within a full length protein.

The terms “bind(s) specifically” or “specifically bind(s)” or “attached” or “attaching” refers to the preferential association of an anti-DEFB-126 antibody, in whole or part, with a cell or tissue bearing a particular target epitope (i.e., a DEFB-126 polypeptide) in comparison to cells or tissues lacking that target epitope. It is, of course, recognized that a certain degree of non-specific interaction may occur between an antibody and a non-target epitope. Nevertheless, specific binding, may be distinguished as mediated through specific recognition of the target epitope. Typically specific binding results in a much stronger association between the delivered molecule and an entity (e.g., an assay well or a cell) bearing the target epitope than between the bound antibody and an entity (e.g., an assay well or a cell) lacking the target epitope. Specific binding typically results in greater than about 10-fold and most preferably greater than 100-fold increase in amount of bound anti-DEFB-126 antibody (per unit time) to a cell or tissue bearing the target epitope as compared to a cell or tissue lacking the target epitope. Specific binding between two entities generally means an affinity of at least 10⁶ M⁻¹. Affinities greater than 10⁸ M⁻¹ are preferred. Specific binding can be determined using any assay for antibody binding known in the art, including Western Blot, ELISA, flow cytometry, immunohistochemistry.

The terms “identical” or percent “identity,” in the context of two or more nucleic acids or polypeptide sequences, refer to two or more sequences or subsequences that are the same or have a specified percentage of amino acid residues or nucleotides that are the same (i.e., share at least about 80% identity, for example, at least about 85%, 90%, 91%, 92%, 93%, 94%, 95%, 96%, 97%, 98%, 99% identity over a specified region to a reference sequence, e.g., SEQ ID NO:4, a polypeptide encoded by SEQ ID NO:6, or the DEFB-126 sequences described herein, when compared and aligned for maximum correspondence over a comparison window, or designated region as measured using one of the following sequence comparison algorithms or by manual alignment and visual inspection. Such sequences are then said to be “substantially identical.” This definition also refers to the compliment of a test sequence. Preferably, the identity exists over a region that is at least about 25 amino acids or nucleotides in length, for example, over a region that is 50-100 amino acids or nucleotides in length, or over the full-length of a reference sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins to DEFB-126 nucleic acids and proteins, the BLAST and BLAST 2.0 algorithms and the default parameters discussed below are used.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., eds., Current Protocols in Molecular Biology (1995 supplement)).

Examples of algorithms that are suitable for determining percent sequence identity and sequence similarity are the BLAST and BLAST 2.0 algorithms, which are described in Altschul et al., J. Mol. Biol. 215:403-410 (1990) and Altschul et al., Nucleic Acids Res. 25:3389-3402 (1977), respectively. Software for performing BLAST analyses is publicly available through the National Center for Biotechnology Information (on the worldwide web at ncbi.nlm.nih.gov/). The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=-2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The term “threshold level” refers to a representative level of binding, e.g., of an anti-DEFB-126 antibody, a lectin, or a poly-cation substance, e.g., to a sperm cell. The threshold level can represent binding detected in a sample from a normal control, a DEFB-126 heterozygous individual or an individual who is homozygous for variant DEFB-126 deletion polymorphism. The threshold level can be determined from an individual or from a population of individuals. In the present diagnostic methods, binding above the threshold level is generally indicative of a likelihood of fertility; binding below the threshold level is generally indicative of an increased risk of infertility.

### BRIEF DESCRIPTION OF THE DRAWINGS

FIG. 1 illustrates the nucleotide (SEQ ID NO:4) and amino acid (SEQ ID NO:6) sequences of wild-type DEFB-126 cDNA. Position of sequence variation (2-nucleotide deletion) indicated by inverse shading. Primary sequence of wild-type carboxy-terminal tail is indicated.

FIG. 2 illustrates the nucleotide (SEQ ID NO:13) and amino acid (SEQ ID NO:16) sequences of the variant DEFB-126 deletion polymorphism cDNA. Position of sequence variation (2-nucleotide deletion) is indicated. Primary sequence of polymorphism variant carboxy-terminal tail is indicated. Note that there is no in-frame stop codon in the newly generated reading frame, resulting in a long poly lysine tail.

FIG. 3 provides a comparison of the C-terminal amino acids sequences (at the forward slash) of both the wild-type (SEQ ID NO:22) and DEFB-126 deletion polymorphism variant (SEQ ID NO:23). The amino acids following the backslash illustrates the difference in the C-terminal sequences between the wild-type and variant DEFB-126 polypeptides.

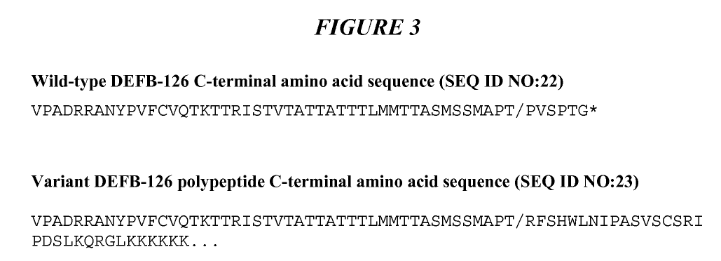

FIG. 4 illustrates a simplified sketch of the domain structure of DEFB 126. Solid lines indicate the presence of 3 disulfide bonds in the β-defensin domain. Diamonds represent the O-linked carbohydrates in the carboxy-terminal domain.

FIG. 5 illustrates the differences between DEFB-126 wild-type and polymorphism genotypes. FIG. 5A illustrates the relative population occurrence of the deletion polymorphism (SEQ ID NOS:24 and 25). FIG. 5B illustrates lowered DEFB-126 mRNA levels in DEFB-126 epididymal tissues. FIG. 5C compares the different protein sequences with associated O-linked glycans in both wild-type and variant DEFB-126 polypeptide. FIG. 5D illustrates the positions of potential O-linked carbohydrate substitutions and cationic amino acid residues in the C-terminal portions of DEFB-126 wild-type and deletion mutant polypeptide sequences (SEQ ID NOS:26 and 27).

FIG. 6 illustrates expression of wild-type and variant DEFB-126 in epididymis. The mRNA encoding aberrant protein is often less stable and present in lower steady state concentrations because of more rapid degradation. Real-time PCR analysis reveals a reduced level of variant DEFB126 (solid bars) compared to wild-type sequence (open bars), when experimental values were normalized to either total input RNA (left panel) or to a control “housekeeping” gene GAPDH (right panel). Also shown are the very high expression of DEFB126 in the epididymis. Two other β-defensins (DEFB125 and DEFB129) are shown for comparison.

FIG. 7 illustrates the differences in fluorescence between sperm labeled with conjugated lectin ABA, which binds structures specific to O-lined glycans.

FIG. 8 illustrates the mass spectrum of O-linked oligosaccharides from DEFB-126 that suggest the highly sialylated nature of DEFB-126.

FIG. 9 illustrates the mass spectrum of glycosylated human defensin-5 (HD %). The mass spectrum of the unglycosylated fraction is compared with the mass spectrum of the glycolsylated form in FIG. 8C.

FIG. 10 illustrates the localized expression of the DEFB-126 polypeptide.

FIG. 11 illustrates sperm penetration of cervical mucus following treatments that mask, modify or remove DEFB-126.

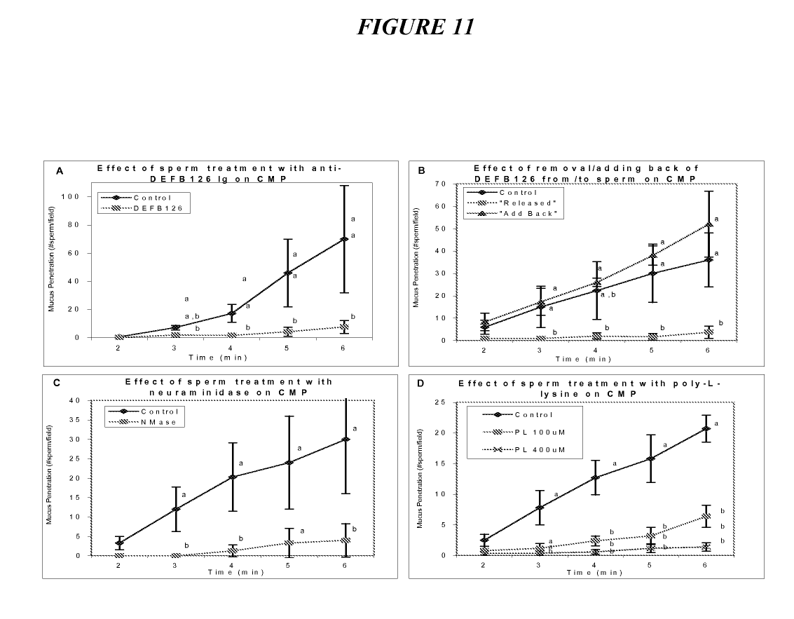

FIG. 12 provides an aligned comparison of the wild-type DEFB-126 protein sequence among the primate species Homo sapiens (SEQ ID NO:6), Hylobates lar (SEQ ID NO:7), Gorilla gorilla (SEQ ID NO:8), Pan troglodytes (SEQ ID NO:9), Macaca fascicularis (SEQ ID NO10), and Pongo pygmaeus (SEQ ID NO:11). Consensus sequence=SEQ ID NO:12).

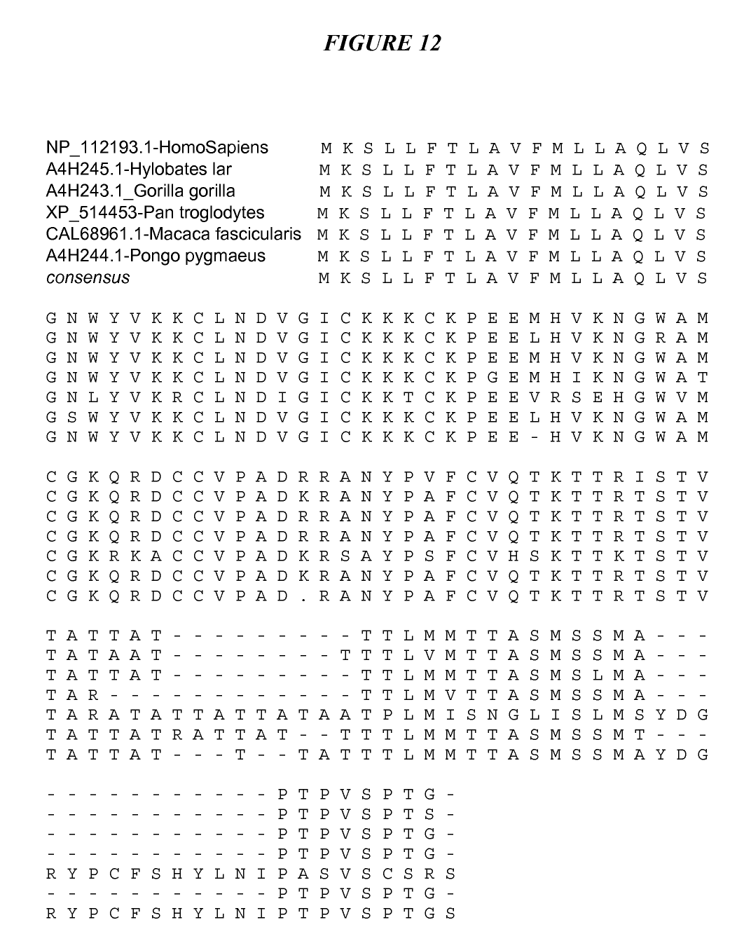

FIG. 13 provides an amino acid sequence diagram of the three domains of Macaca fascicularis DEFB-126 (GenBank Accession No. Q9BEE3) (SEQ ID NO:28): (1) the signal sequence (aa 1-20), (2) the β-defensin core region (aa 21-64), and (3) the carboxyl tail (aa 65-123).

FIG. 14 provides a diagram of the sperm penetration of Cervical Mucus (CM) or HA gels.

FIG. 15 provides data describing the ability of human sperm exhibiting the DEBF126 polymorphism and to penetrate HA gel (15A). Sperm suspensions were analyzed by CASA for average curvilinear velocity (VCL) (15B). Slides with smears of sperm suspensions were “Pap”-stained and analyzed according to WHO ‘89 sperm morphology method and reported as total average percent normal forms (% normal) (15C). ABA lectin labeling outcomes were averaged across sperm from donors possessing wt DEFB126 gene (wt=wt/wt+wt/del) and sperm from donors that possessed only the gene variant (del/del=del) (D).

FIG. 16 provides data demonstrating differences in FITC-conjugated lectin ABA labeling of human sperm from wt and del donors, as previously described in FIG. 7 and 15D.

FIG. 17 provides data describing HA penetration with sperm from del donor D10 and wt donor D12. HA penetration of sperm from donor D10 (dark gray squares) and donor D12 (light gray squares) are shown in reference to average values for del and wt males, respectively.

FIG. 18 provides data describing that DEFB126 can be “added back” to the sperm surface.

FIG. 19 provides data describing treatment of sperm from del males with cDEFB 126 improved sperm penetration of HA Gel. Plot represents mean±sd response of sperm from 3 different del/del donors (19A). Plot represents mean±sd response of sperm from 2 del/del donors that showed ˜4-fold increase in penetration rate with addition of cDEF126 (19B).

FIG. 20 provides BLAST amino acid analysis comparison of cynomolgus macaque and human DEFB-126 showing that the functional DEFB-126 proteins of these two primate species only share 71% sequence homology over the positions compared by the algorithm, positions 1-134 of cynomolgus macaque DEFB-126 and 1-121 of human DEFB-126. (FIG. 20A) (SEQ ID NOS:29 and 30; consensus=SEQ ID NO:31). Eliminating the signal sequence drops the homology to 66% (FIG. 20B) (SEQ ID NOS:32 and 33; consensus=SEQ ID NO:34).

### DETAILED DESCRIPTION

I. Introduction

The present invention is based, in part, on the unexpected discovery that individuals possessing a common DEFB-126 deletion polymorphism show an increased risk or likelihood of infertility in comparison to individuals having a wild-type DEFB-126 genotype. Genetic polymorphisms can provide a useful way in which to distinguish different alleles of a gene. Furthermore, when the presence of a polymorphism can be associated with a specific phenotype, the polymorphism operates as a powerful marker and can be used to determine phenotypic outcomes based on an individual's genotypic makeup.

In particular, the present invention relates to a dinucleotide deletion polymorphism in a DEFB-126 nucleic acid sequence. The amino acid sequence of this variant has a significantly altered the carboxyl terminal, carbohydrate-containing domain of DEFB-126. This variant results in aberrant DEFB-126 protein function and structure, leading to reduced sperm function and fertility. By identifying individuals possessing a DEFB-126 deletion polymorphism early in an infertility evaluation, clinicians can obtain scientific evidence to justify rapid progression to directed interventions such as intrauterine insemination (IUI) and in vitro fertilization (IVF), thus saving couples the time and expense of a protracted workup.

Mature sperm released from the male tract at ejaculation must spend time in the female tract before they are competent to fertilize. This final maturation process, termed capacitation, has been recognized for more than fifty years as an essential prerequisite for fertilization. It has recently been demonstrated in the cynomolgus monkey (Macaca fascicularis) that a single epididymis-derived protein forms a continuous coat on sperm that remains tightly adhered to sperm even after rigorous washing through gradient solutions, but is then released from the sperm surface during in vitro capacitation (Tollner et al., Mol. Reprod. Dev. 69:325-337 (2004); Yudin et al., Biol. Reprod. 73:1243-1252 (2005); Yudin et al., J. Membr. Biol. 207:119-129 (2005); Yudin et al., Biol. Reprod. 69:1118-1128 (2003)). This epididymal secretory protein was originally called ESP 13.2, but is now called DEFB-126 due to its amino acid sequence homology and structural similarity to β-defensins (Lehrer et al., Mucosal Immunology, 3^{rd} Ed., Academic Press: New York, 95-110 (2004)). β-defensins are antimicrobial proteins that disrupt target membranes (see, e.g., Diamond and Bevins, Clinical Immunology and Immunopathology 88:221-25 (1998)).

DEFB-126 is highly glycosylated on its COOH-tail with O-linked sialic acid (Yudin et al., J. Membr. Biol. 207:119-129 (2005)). Viable sperm recovered from the cervix and uterus of mated female macaques are evenly coated with DEFB-126 over the entire surface suggesting that DEFB-126 is retained on sperm in the upper female reproductive tract Tollner et al., Biol. Reprod. 78:400-412 (2008)). DEFB-126 may provide an immunoprotective shield, which could block sperm surface recognition by the female reproductive tract, thereby assuring a safe haven for sperm storage and ultimate capacitation. (Yudin et al., Biol. Reprod. 73:1243-1252 (2005)). Men possessing a DEFB-126 allele with the dinucleotide deletion polymorphism and expressing a variant DEFB-126 polypeptide produce sperm that, although apparently normal, are dysfunctional in the female environment.

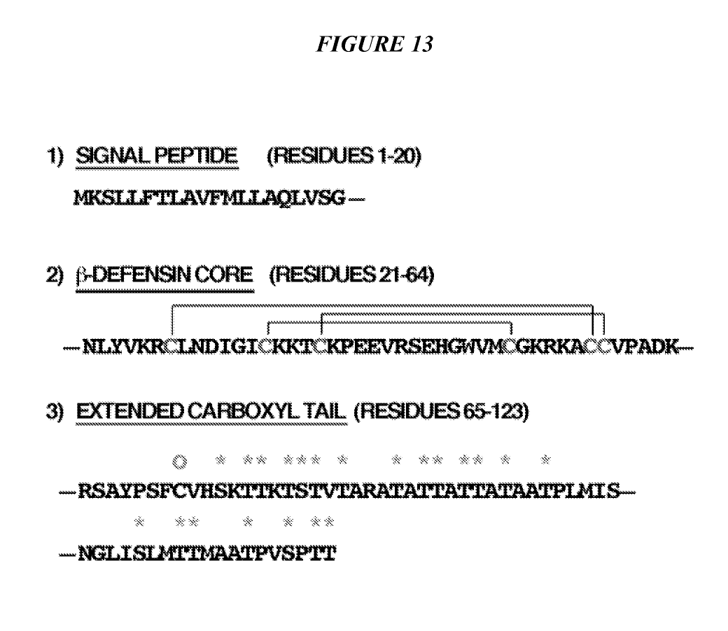

The amino acid sequence of DEFB126 reveals a signal sequence (aa 1-20) and a 45 amino acid β-defensin domain (aa 21-64). Like other β-defensins, DEFB126 has a specific six-cysteine organization (Schutte et al., 2002). The 60 amino acid C-terminal domain has an unpaired cysteine (open circle) and numerous potential sites (*) for O-linked glycosylation (Julenius et al., 2005) (see, FIG. 13).

II. Determining the Risk of Reduced Male Fertility by Identifying the DEFB-126 Deletion Polymorphism

The present invention provides methods for analyzing the genotype of individuals with respect to the gene encoding DEFB-126 in order to determine whether that individual has reduced fertility. Such determination will provide an individual knowledge of whether their genotype is associated with a risk or likelihood of reduced fertility, thereby allowing that individual to receive appropriate fertility treatment options. The present invention further provides kits that are useful for diagnosing increased risk of infertility based on the presence or absence of the DEFB-126 deletion polymorphism.

DEFB-126 Deletion Polymorphisms Associated with Reduced Fertility

Capacitation is the final sperm maturation process, which is essential for fertilization. During this process, a single epididymis-derived protein that forms a continuous coat on sperm is released. This protein was originally called ESP 13.2 (epididymal secretory protein), but is now called DEFB-126 based on its amino acid sequence homology and structural similarity to β-defensins (Lehrer et al., Mucosal Immunology, 3^{rd} Ed., Academic Press: 95-110, (2004); Diamond and Bevins, Clin. Immunol. Immunopathol. 88:221-25 (1998)). DEFB-126 is highly glycosylated on its COOH-tail with O-linked sialic acid (Yudin et al., J. Membr. Biol. 207:119-29 (2005)). DEFB-126 is thought to provide an immunoprotective shield from sperm surface recognition by the female reproductive tract prior to capacitation (Yudin et al., Biol. Reprod. 73:1243-1252 (2005)).

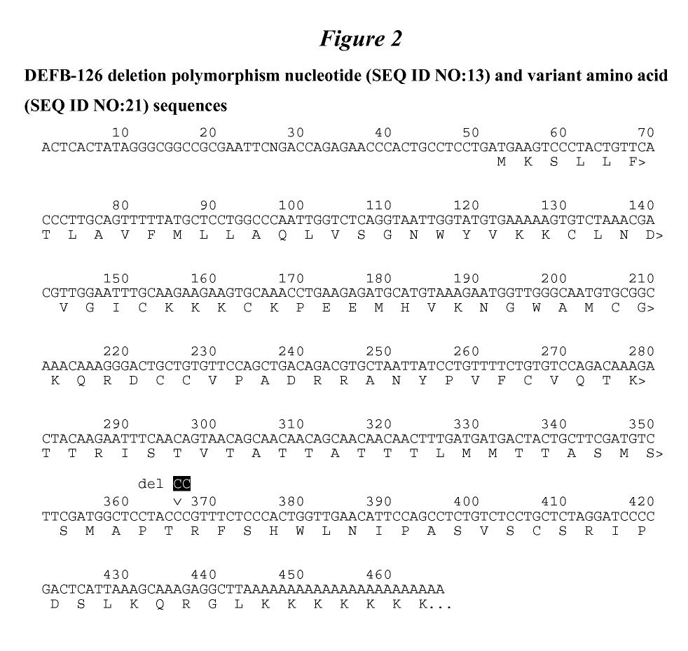

The DEFB-126 deletion polymorphism associated with increased infertility is a two-nucleotide “CC” deletion within the contextual nucleic acid subsequence of a wild-type DEFB-126 nucleotide sequence defined by TCCTACCCCCGTTTC (SEQ ID NO:1). The DEFB-126 deletion polymorphism will not contain more than three cytosines within positions 6-10 of the contextual subsequence TCCTACCCCCGTTTC of SEQ ID NO:1 (see also FIG. 2). Exemplary DEFB-126 nucleic acid sequences having the deletion polymorphism have the nucleotide sequence provided in SEQ ID NO:13 (GenBank Accession No. AK225987), SEQ ID NO:14, and SEQ ID NO:15 (GenBank Accession No. CO408416). When this deletion polymorphism is present, there are no stop codons in the newly generated reading frame, which results in an extended poly-lysine tail. Whereas the wild-type DEFB-126 protein provides amino acid residues for O-linked carbohydrates that bring a negative charge to the C-terminal domain, the poly-lysine tail in DEFB-126 mutants brings a more positive charge to this domain.

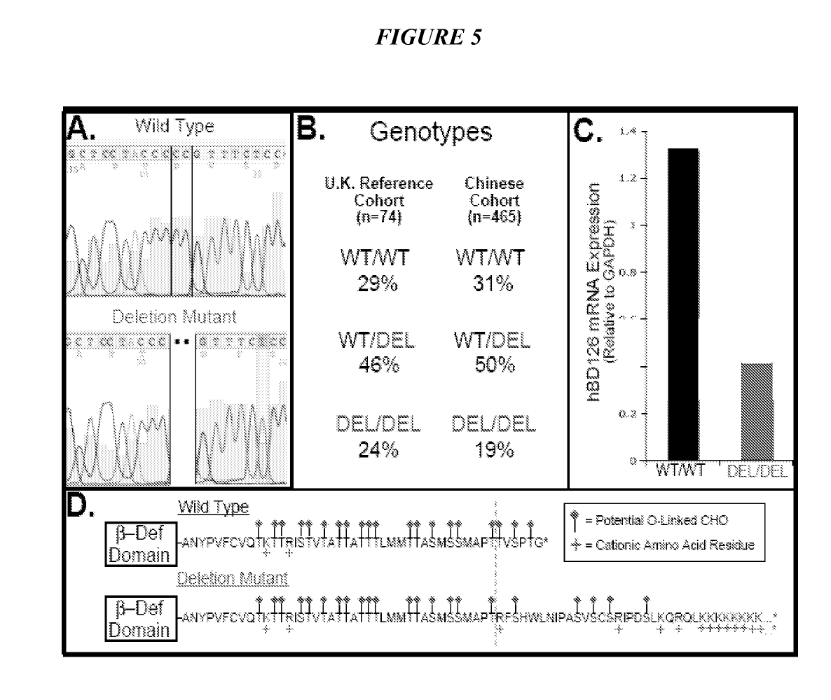

A sequence variation in DEFB 126 cDNA that has 2-nucleotide omission (deletion) which causes a frame-shift in the open reading frame has also been identified (see, FIG. 5A). The allele was identified by genotype analysis of a total of 465 randomly selected individuals from a cohort of Chinese men (collaboration with S. Venners & Xiping Xu, U. Chicago) and 74 individuals from a population of men in Great Britain (see, FIG. 5B). mRNA encoding aberrant protein is often present in tissue at lower steady state concentrations because of more rapid degradation. Therefore, we analyzed DEFB126 mRNA in epididymal tissue using quantitative RT-PCR (qPCR). A reduced level of DEFB126 mRNA was observed in an epididymal specimen with the sequence variant, consistent with this prediction (see, FIG. 5C).

A sequence variation in DEFB 126 cDNA that has 2-nucleotide omission (deletion) which causes a frame-shift in the open reading frame has also been identified (see, FIG. 5). This specific DEFB126 polymorphism has allele frequency of approximately ˜0.45-0.50. The variant amino acid sequence of the variant DEFB126 predicts a significantly altered C-terminal, carbohydrate-containing domain. For this variant, as the open reading frame would extend into the polyA tail, the variant is likely a null (non-expressing) allele. Furthermore, epididymal tissue in individuals with the DEFB-126 deletion polymorphism have markedly lower expression of DEFB-126 mRNA when compared to wild-type epididymal tissue, as mRNA encoding aberrant protein is often present in lower steady state concentrations as a result of rapid degradation (see, FIG. 5). The DEFB126 variant had a 2-nucleotide omission (deletion), causing a frame-shift in the open reading frame of DEFB126 (FIG. 5A). This sequence variation was confirmed in the NCBI genomic DNA sequence database.

### Nucleic Acid Detection of DEFB-126 Deletion Polymorphisms

The DEFB-126 deletion polymorphism can be detected using any methods known in art, including without limitation amplification, sequencing and hybridization techniques. Detection techniques for evaluating nucleic acids for the presence of a single base change involve procedures well known in the field of molecular genetics. Methods for amplifying nucleic acids find use in carrying out the present methods. Ample guidance for performing the methods is provided in the art. Exemplary references include manuals such as PCR Technology: Principles And Applications For DNA Amplification (ed. H. A. Erlich, Freeman Press, New York, N.Y., (1992)); PCR Protocols: A Guide To Methods And Applications (Innis, et al., eds., Academic Press, San Diego, Calif., (1990)); Current Protocols In Molecular Biology, Ausubel, (1990-2008, including supplemental updates); Sambrook & Russell, Molecular Cloning, A Laboratory Manual (3rd Ed, 2001).

According to one aspect of the present invention, the DEFB-126 deletion polymorphism is detected by an amplification reaction. The DEFB-126 region is amplified using an oligonucleotide pair to form nucleic acid amplification products of DEFB-126 deletion polymorphism sequences. Amplification can be by any of a number of methods known to those skilled in the art including PCR, and the invention is intended to encompass any suitable methods of DNA amplification. A number of DNA amplification techniques are suitable for use with the present invention. Conveniently such amplification techniques include methods such as polymerase chain reaction (PCR), strand displacement amplification (SDA), nucleic acid sequence based amplification (NASBA), rolling circle amplification, T7 polymerase mediated amplification, T3 polymerase mediated amplification and SP6 polymerase mediated amplification. The precise method of DNA amplification is not intended to be limiting, and other methods not listed here will be apparent to those skilled in the art and their use is within the scope of the invention.

In some embodiments, the polymerase chain reaction (PCR) process is used (see, e.g., U.S. Pat. Nos. 4,683,195 and 4,683,202. PCR involves the use of a thermostable DNA polymerase, known sequences as primers, and heating cycles, which separate the replicating deoxyribonucleic acid (DNA), strands and exponentially amplify a gene of interest. Any type of PCR, including quantitative PCR, RT-PCR, hot start PCR, LA-PCR, multiplex PCR, touchdown PCR, finds use. In some embodiments, real-time PCR is used.

The amplification products are then analyzed in order to detect the presence or absence of the DEFB-126 deletion polymorphism that is associated with reduced fertility. By practicing the methods of the present invention and analyzing the amplification products it is possible to determine whether the individual being tested is at a risk of reduced fertility.

In some embodiments, analysis may be made by restriction fragment length polymorphism (RFLP) analysis of a PCR amplicon produced by amplification of genomic DNA with the oligonucleotide pair. In order to simplify detection of the amplification products and the restriction fragments, those of skill will appreciate that the amplified DNA will further comprise labeled moieties to permit detection of relatively small amounts of product. A variety of moieties are well known to those skilled in the art and include such labeling tags as fluorescent, bioluminescent, chemiluminescent, and radioactive or colorigenic moieties.

A variety of methods of detecting the presence and restriction digestion properties of DEFB-126 gene amplification products are also suitable for use with the present invention. These can include methods such as gel electrophoresis, mass spectroscopy or the like. The present invention is also adapted to the use of single stranded DNA detection techniques such as fluorescence resonance energy transfer (FRET). For FRET analysis, hybridization anchor and detection probes may be used to hybridize to the amplification products. The probes sequences are selected such that in the presence of the polymorphism, for example, the resulting hybridization complex is more stable than if there is a G or C residue at a particular nucleotide position. By adjusting the hybridization conditions, it is therefore possible to distinguish between individuals with the DEFB-126 deletion polymorphism and those without. A variety of parameters well known to those skilled in the art can be used to affect the ability of a hybridization complex to form. These include changes in temperature, ionic concentration, or the inclusion of chemical constituents like formamide that decrease complex stability. It is further possible to distinguish individuals heterozygous for the DEFB-126 deletion polymorphism versus those that are homozygous for the same. The method of FRET analysis is well known to the art, and the conditions under which the presence or absence of the DEFB-126 deletion polymorphism would be detected by FRET are readily determinable.

Suitable sequence methods of detection also include e.g., dideoxy sequencing-based methods and Maxam and Gilbert sequencing (see, e.g., Sambrook and Russell, supra). Suitable HPLC-based analyses include, e.g., denaturing HPLC (dHPLC) as described in e.g., Premstaller and Oefner, LC-GC Europe 1-9 (July 2002); Bennet et al., BMC Genetics 2:17 (2001); Schrimi et al., Biotechniques 28(4):740 (2000); and Nairz et al., PNAS USA 99(16):10575-10580 (2002); and ion-pair reversed phase HPLC-electrospray ionization mass spectrometry (ICEMS) as described in e.g., Oberacher et al.; Hum. Mutat. 21(1):86 (2003). Other methods for characterizing DEFB-126 alleles include, e.g., single base extensions (see, e.g., Kobayashi et al, Mol. Cell. Probes, 9:175-182 (1995)); single-strand conformation polymorphism analysis, as described, e.g, in Orita et al., Proc. Nat. Acad. Sci. 86, 2766-2770 (1989), allele specific oligonucleotide hybridization (ASO) (e.g., Stoneking et al., Am. J. Hum. Genet. 48:70-382 (1991); Saiki et al., Nature 324:163-166 (1986); EP 235,726; and WO 89/11548); and sequence-specific amplification or primer extension methods as described in, for example, WO 93/22456; U.S. Pat. Nos. 5,137,806; 5,595,890; 5,639,611; and U.S. Pat. No. 4,851,331; 5′-nuclease assays, as described in U.S. Pat. Nos. 5,210,015; 5,487,972; and 5,804,375; and Holland et al., 1988, Proc. Natl. Acad. Sci. USA 88:7276-7280 (1988).

Exemplary polynucleotides for use in detecting a wild-type or variant DEFB-126 polynucleotide are summarized in the following table:

| Name | Forward/Reverse | Sequence (SEQ ID NO:) |
|---|---|---|
| DEFB126-154s | forward | AAG AAT GGT TGG GCA ATG TGC (36) |
| DEFB126-199s | forward | GCA AAC AAA GGG ACT GCT GTG TTC C (37) |
| DEFB126-330a | reverse | AGG AGC CAT CGA AGA CAT CGA AGC (38) |
| DEFB126-409a | reverse | CCA CAA TGC TTT AAT GAG TCG GG (39) |
| DEFB126-278s | forward | CAG CAA CAA CAA CTT TGA TGA TGA C (40) |
| DEFB129-441s | forward | CCA TCA GCA CTA TGA CCC CAG GAC (41) |
| DEFB129-546a | reverse | GTT GGC AGT ATG TTT GGT GGA GGT G (42) |

For example, the methods can employ a forward primer selected from the group consisting of DEFB126-154s, DEFB126-199s, DEFB126-278s and DEFB129-441s and a reverse primer selected from the group consisting of DEFB126-330a, DEFB126-409a and DEFB129-546a. In some embodiments, the primers DEFB126-154s and DEFB126-409a are used to determine the DEFB-126 genotype of an individual, e.g., by DNA sequence analysis of the amplification product.

In some embodiments, the methods employ the polynucleotide DEFB126-278s, which finds use as a sequencing primer to determine a DEFB-126 genotype, e.g., by DNA sequence analysis of the amplification product.

The polynucleotides can be labeled for detection using methods known in the art and as described herein.

### Detection of Proteins Expressed by DEFB-126 Deletion Polymorphisms

The DEFB-126 deletion polymorphism can be detected using any methods known in the art. For example, DEFB-126 wild-type and variant proteins can be detected by analyzing the physical differences between the protein products of the expressed wild-type and polymorphic DEFB-126 genes.

One physical difference between the wild-type and variant DEFB-126 polypeptides is the relative abundance of the protein. The DEFB-126 deletion polymorphism mRNA is often present in lower steady state concentrations, and often observed with aberrantly expressed mRNA sequences. This results in an overall lower abundance (i.e., reduced presence) of the variant DEFB-126 polypeptide in comparison to wild-type DEFB-126 polypeptide. In some embodiments, the variant DEFB-126 polypeptide is completely absent or undetectable. When detectable, the variant DEFB-126 polypeptide may be present in amounts that are about 50%, 30%, 10%, or less in comparison to detectable amounts of the wild-type DEFB-126 polypeptide.

As described above, when expressed, the variant DEFB-126 polypeptide contains an extended C-terminal domain resulting from a read through of the wild-type stop codon as a result of the nucleotide sequence frame shift. This extended C-terminal variant DEFB-126 polypeptide provides an observable physical difference between the wild-type and variant DEFB-126 polypeptides. A wild-type DEFB-126 polypeptide has 111 amino acid residues, whereas the variant DEFB-126 polypeptide contains at least 132 amino acid residues. In addition to containing an extended C-terminus, the variant DEFB-126 polypeptide has a significant reduction in C-terminal O-linked glycosylation.

The molecular weight and isoelectric point of the variant DEFB-126 polypeptide provide notable physical differences between the variant and wild-type DEFB-126 polypeptides. The predicted molecular weight of the unglycosylated variant DEFB-126 polypeptide (SEQ ID NO:16 and SEQ ID NO:17) based on deduced amino acid sequence is at least about 11.53 kDa and the isoelectric point is at least pH 10.75. In contrast, the predicted molecular weight of an unglycosylated wild-type DEFB-126 polypeptide (SEQ ID NO:6) based on deduced amino acid sequence is about 9.19 kDa, and the isoelectric point is approximately pH 10.75. In non-human primates, the non-reduced and reduced native DEFB-126 protein has an apparent molecular weight in polyacrylamide gels of about 53 kDa (protein band spans 51-55 kDa) and about 34 kDa (protein band spans 31-36 kDa), respectively.

Detection of the variant DEFB-126 polypeptide may be accomplished by an antibody. For use as an antigen for the development of antibodies, the DEFB-126 protein naturally produced or expressed in recombinant form or a functional derivative thereof, preferably having at least 9 amino-acids, is obtained and used to immunize an animal for production of a polyclonal or monoclonal antibody. An antibody is said to be capable of binding a molecule if it is capable of reacting with the molecule to thereby bind the molecule to the antibody. The specific reaction is meant to indicate that the antigen will react in a highly selective manner with its corresponding antibody and not with the multitude of other antibodies which may be evoked by other antigens.

The term antibody herein includes but is not limited to human and non-human polyclonal antibodies, human and non-human monoclonal antibodies (mAbs), chimeric antibodies, anti-idiotypic antibodies (anti-IdAb) and humanized antibodies. Polyclonal antibodies are heterogenous populations of antibody molecules derived either from sera of animals immunized with an antigen or from chicken eggs. Monoclonal antibodies (mAbs“) are substantially homogenous populations of antibodies to specific antigens. mAbs may be obtained by methods known to those skilled in the art (e.g., U.S. Pat. No. 4,376,110). Such antibodies may be of any immunological class including IgG, IgM, IgE, IgA, IgD and any subclass thereof. The hybridoma producing human and non-human antibodies to DEFB-126 may be cultivated in vitro or in vivo. For production of a large amount of mAbs, in vivo is the presently preferred method of production. Briefly, cells from the individual hybridomas are injected intraperitoneally into pristine primed Balb/x mice or Nude mice to produce ascites fluid containing high concentrations of the desired mAbs. MAbs may be purified from such ascites fluids or from culture supernatants using standard chromatography methods well known to those of skill in the art (see, e.g. E. Harlow, Antibodies: A Laboratory Manual, Cold Spring Harbor Laboratory Press, Cold Spring Harbor, N.Y. (1988)).

The antibodies or fragments of antibodies are used to quantitatively or qualitatively detect the presence of either the wild-type of variant DEFB-126 polypeptide in biological samples obtained as described above. Because the wild-type and variant DEFB-126 polypeptides differ at the C-terminus, antibodies can be developed that specifically bind to either the wild-type or variant DEFB-126 C-terminus. In some embodiments, the distinguishing antibody specifically binds to an epitope within the C-terminus of a wild-type DEFB-126 polypeptide. In some embodiments, the distinguishing antibody specifically binds to an epitope within the C-terminus of a variant DEFB-126 polypeptide.

Detection of the presence of the variant DEFB-126 polypeptide may be accomplished by binding of the polypeptide to a lectin, a polycation (e.g., poly-L-lysine) or an antibody generated as described above. The biological sample may be treated with a solid phase support or carrier such as nitrocellulose or other solid supports capable of immobilizing cells or cell particles or soluble proteins. The support may then be washed followed by treatment with the detectably labeled anti-DEFB-126 antibody. This is followed by wash of the support to remove unbound antibody. The term solid phase support refers to any support capable of binding antigen or antibodies including, but not limited to, glass, polystyrene polypropylene, nylon, modified cellulose, or polyacrylamide. The amount of bound label on said support may then be detected by conventional means including, but not limited to, Western blot hybridization, ELISA, or immunoprecipitation.

Detection using a lectin, a polycation (e.g., poly-L-lysine) or an antibody may further be accomplished by immunofluorescence techniques employing a fluorescently labeled antibody with a fluorescent microscopy, light microscopy, immunoelectron microscopy, in situ hybridization, flow cytometric or fluorometric detection. The abovementioned techniques used to detect the presence of a wild-type or variant DEFB-126 polypeptide using an antibody are representative examples of various techniques well known in the art, but should not be limited only to those described above.

Detection of the variant DEFB-126 polypeptide may be accomplished using a lectin that selectively binds Galactose-GalNAc or sialic acid. For example, the lectins Agaricus bisporus (ABA) or Artocarpus integrifolia (Jacalin) selectively bind galactose-GalNAc-serine (or threonine), which are structures specific to O-linked glycans. Sperm from individuals possessing the variant DEFB-126 polypeptide show significant reduction in ABA-associated fluorescence compared to wild-type donors. Significant reduction in sialic acid moieties on the sperm surface has been demonstrated with loss of DEFB 126 from non-human primate sperm. Reduced binding levels of lectins which recognizes sialic acid moieties in terminal positions on oligosaccharides on DEFB126 in comparison to a normal control or a predetermined threshold level is therefore indicative of increased risk of infertility. The lectins Limulus polyphemus (LPA), Macackia amurenesis (MAL II), or Triticum vulgaris (WGA) can be used to detect sialic acid moieties. In some embodiments, lectin detection is accomplished by treating sperm with neuraminidase, fixing with paragormaldehyde/gluteraldehyde, and then incubating the sperm with FITC-conjugated lectin ABA.

Detection of the variant DEFB-126 polypeptide also may be accomplished using poly-L-lysine (or similar polycationic substance), which binds to negatively charged oligosaccharide moieties associated with DEFB-126. Removal of DEFB-126 from sperm with activator compounds (ACT) or treatment of sperm with sialidase or O-glycosidase results in significant loss of binding of poly-L-lysine to the surface of non-human primate sperm. Yudin et al. J. Membrane Biol. 207, 119-129 (2005). Reduced binding levels of poly-L-lysine, which recognizes negatively charged moieties associated with oligosaccharides on DEFB126, in comparison to normal control is therefore indicative of increased risk of infertility

Lectins, polycations (e.g., poly-L-lysine) and/or antibodies can be used in conjunction with available technologies employed in diagnostic kits for the detection of the DEFB126 glycoprotein in a semen sample. The diagnostic kits could be for use in the clinic or in a home setting. Differences in sperm labeling can be examined and threshold values can discriminate between DEFB126 wild-type or “positive” and DEFB126 deletion variant or “negative” males. Threshold values can determined, e.g., by comparison to a DEFB126 positive or negative control. The threshold value can be determined by evaluating lectin, poly-L-lysine (or similar polycationic substance) and/or antibody binding to a population of individuals known to possess either the wild-type or variant form of DEFB126. If the lectin, poly-L-lysine (or similar polycationic substance) or antibody binding in the test sample is similar or equivalent to lectin, poly-L-lysine (or similar polycationic substance) or antibody binding in a negative control or a control sample representative of the DEFB126 variant form, then the test sample indicates presence of the DEFB126 variant form and the likelihood of infertility. If the lectin, poly-L-lysine (or similar polycationic substance) or antibody binding in the test sample is similar or equivalent to lectin, poly-L-lysine (or similar polycationic substance) or antibody binding in a positive control or a control sample representative of the DEFB 126 wild-type form, then the test sample indicates presence of the DEFB 126 wild-type form and diminished likelihood of infertility or infertility for a reason other than a variant DEFB126. If the lectin, poly-L-lysine (or similar polycationic substance) or antibody binding in the test sample is less than lectin, poly-L-lysine (or similar polycationic substance) or antibody binding in a positive control or a control sample representative of the DEFB126 wild-type form, then the test sample indicates presence of the DEFB126 variant form and the likelihood of infertility.

Lectins of use selectively bind Galactose-GalNAc or sialic acid. It has been described in the macaque model that lectin ABA as well as wheat germ agglutinin (WGA) strongly recognize DEFB 126 on intact (and viable) sperm and on Western blots (Yudin et al., (2005)). Labeling with ABA requires that sperm initially be treated with sialidase, a step that can be performed with both living and fixed sperm (Yudin et al., (2005); Tollner et al., (2008)).

The lectins however bind to the same classes of oligosaccharides that are potentially associated with glycolipids and other glyoproteins of the sperm glycocalyx. Similarly, poly-L-lysine (or similar polycationic substance) will bind to highly negatively charged moieties potentially associated with other biomolecules on the sperm surface. As such, antibodies can be used in conjunction with lectins or poly-L-lysine (or similar polycationic substance) to provide for greater specificity if required. Antibodies to sperm-specific proteins have been used to estimate sperm concentration using a lateral flow immunochromatographic home test device (“SPERMCHECK®”; Klotz et al., 2008). In some embodiments, lectins, poly-L-lysine (or similar polycationic substance), or antibodies that specifically bind to DEFB126 are used for infertility diagnoses. In some embodiments, lectins, or poly-L-lysine (or similar polycationic substance) can be used in combination with sperm-specific antibodies for infertility analyses.

Detection of the variant DEFB-126 polypeptide may be accomplished by determination of the physical and/or chemical properties of the wild-type and variant DEFB-126 polypeptides as presented above. Such properties include the molecular weight and isoelectric point, the sequence of the DEFB-126 polypeptide's C-terminus, and the degree of glycosylation of the DEFB-126 C-terminus. Conditions for molecular weight determination and isoelectric point determination are well known in the art, and are described in detail, e.g., in U.S. patent application Ser. No. 07/919,784. Determination of the C-terminal protein sequence may be achieved by any method well known in the art, e.g., the Edman degradation technique (Creighton, Proteins: Structures and Molecular Principles, W. H. Freeman, New York, p. 3449 (1983)). C-terminal glycosylation can be determined using any method known in the art, e.g., as described in Example 4 below.

III. Kits for Identifying DEFB-126 Deletion Polymorphisms

The invention further provides diagnostic kits useful for determining whether an individual possessing the DEFB-126 deletion polymorphism. The kits can contain polynucleotides for use in analyzing an individual's genotypic makeup at the DEFB-126 locus and/or antibodies and/or lectins for use in analyzing an individual's DEFB-126 protein product. The kits can contain polypeptides for use in treatment of an individual with a DEFB-126 polypeptide deletion or mutation or non-expressing allele.

a. Polynucleotides

In general, each of the kits that test an individual's genotypic makeup comprises one or more polynucleotides, e.g., primer pairs suitable to amplify the portions of the gene comprising the DEFB-126 deletion polymorphism of the present invention or probes that selectively hybridize to a wild-type or variant DEFB-126 nucleic acid. In some embodiments, the kits comprise forward and reverse primers suitable for amplification of a genomic DNA sample taken from an individual, and instructions for use. As described above, the biological sample can be from any tissue or fluid in which genomic DNA is present. Conveniently, the sample may be taken from blood, skin or a hair bulb.

The kits contain instructions on how to use a biological sample to generate a template for use in the amplification reaction, and how to use the provided primer sets for optimal amplification reactions. The instructions further describe interpretations of the amplification reaction, and indicate that the presence of five contiguous cytosines “CCCCC” at positions 6-10 of the subsequence TCCTACCCCCGTTTCT (SEQ ID NO:1) is indicative of normal fertility, and the presence of at most three contiguous cytosines “CCC” within positions 6-10 of the above-mentioned subsequence is indicative of an increased risk or probability of infertility.

Exemplary polynucleotides for use in detecting a wild-type or variant DEFB-126 polynucleotide and for inclusion in the kits are summarized in the following table:

| Name | Forward/Reverse | Sequence (SEQ ID NO:) |
|---|---|---|
| DEFB126-154s | forward | AAG AAT GGT TGG GCA ATG TGC (36) |
| DEFB126-199s | forward | GCA AAC AAA GGG ACT GCT GTG TTC C (37) |
| DEFB126-330a | reverse | AGG AGC CAT CGA AGA CAT CGA AGC (38) |
| DEFB126-409a | reverse | CCA CAA TGC TTT AAT GAG TCG GG (39) |
| DEFB126-278s | forward | CAG CAA CAA CAA CTT TGA TGA TGA C (40) |
| DEFB129-441s | forward | CCA TCA GCA CTA TGA CCC CAG GAC (41) |
| DEFB129-546a | reverse | GTT GGC AGT ATG TTT GGT GGA GGT G (42) |

For example, the kits can contain a forward primer selected from the group consisting of DEFB126-154s, DEFB126-199s, DEFB126-278s and DEFB129-441s and a reverse primer selected from the group consisting of DEFB126-330a, DEFB126-409a and DEFB129-546a. In some embodiments, the kits contain the primers DEFB126-154s and DEFB126-409a. The amplification can be used as a template to determine genotype by direct sequence analysis.

In some embodiments, the kits contain primer DEFB126-278s, which finds use as a sequencing primer to determine a DEFB-126 genotype, e.g., by sequence analysis of the amplification product.

The polynucleotides can be labeled for detection using methods known in the art and as described herein.

b. Antibodies

In general, each of the kits that test whether an individual is producing a wild-type or variant DEFB-126 polypeptide comprises at least one antibody that distinguishes between DEFB-126 polypeptides as described above. The kits further provide instructions for use, and the necessary components to run the desired reaction, e.g. polyacrylamide gels, secondary antibodies, buffers, etc.

The kit can also contain instructions on how to use a biological sample with the antibodies supplied and the other components necessary to run the desired reaction. The kit further provides instructions that interpret the presence of a DEFB-126 polypeptide as indicative of normal fertility and the absence or reduced presence of a DEFB-126 polypeptide as indicative of an increased risk of infertility. The detected presence of DEFB-126 can also be determined with reference to a predetermined threshold level. In such embodiments, the kit further provides instructions for interpreting the presence of a DEFB-126 polypeptide above the threshold level as indicative of normal fertility and the absence or presence of a DEFB-126 polypeptide below the threshold level as indicative of an increased risk of infertility. In some embodiments, the antibodies contained in the kits selectively bind to the C-terminus of a wild-type or variant DEFB-126 polypeptide. Additional antibodies that find use can distinguish between wild-type and mutant forms of DEFB-126. In some embodiments, the anti-DEFB-126 antibodies are labeled, e.g., with a fluorophore, a chromophore, a chemiluminiscent moiety, an enzyme, a radioactive isotope, etc.. In some embodiments, the kits contain labeled secondary antibodies.

c. Lectins

In some embodiments, a lectin-DEFB126-antibody “sandwich” approach could also be employed and such an approach would be highly adaptable to a test kit, where the lectins could be employed as a “sperm capture” strategy. For example, an appropriate lectin bound to a solid support can bind sperm via oligosaccharides on wild-type DEFB126. Variant forms of DEFB 126 are not bound by the lectin or are bound at reduced levels. DEFB 126 can be released from the sperm surface by treatment with phospholipase C or conditions of high salt and pH (Yudin et al., (2003); Tollner et al., (2009)). Mono- or polyclonal antibodies conjugated to biotin or enzymes could be applied to the solid phase for colorimetric detection of DEFB126. Similar sandwich assays have greatly enhanced the sensitivity and specificity of detection of serum mucins associated with pancreatic cancer by a monoclonal antibody (Neil Parker, (1998)), and such methods could be easily adapted to the methods of the present invention.

The kit can also contain instructions on how to use a biological sample with the lectins supplied and the other components necessary to run the desired reaction. The kit further provides instructions that interpret the detectable presence of a DEFB-126 polypeptide as indicative of normal fertility and the absence or reduced presence of a DEFB-126 polypeptide as indicative of an increased risk of infertility. The detected presence of DEFB-126 can also be determined with reference to a predetermined threshold level. In such embodiments, the kit further provides instructions that interpret the presence of a DEFB-126 polypeptide above the threshold level as indicative of normal fertility and the absence or presence of a DEFB-126 polypeptide below the threshold level as indicative of an increased risk of infertility. In some embodiments, the kits contain at least one lectin that selectively binds galactose-GalNAc or sialic acid. In some embodiments, the lectin is Agaricus bisporus (ABA) or Artocarpus integrifolia (Jacalin). In some embodiments, the lectin is Limulus polphemus (LPA), Macackia amurenesis (MAL II), or Triticum vulgaris (WGA). In some embodiments, the kits also contain sperm-specific antibodies.

In some embodiments, the lectin comprises a detectable label, e.g., with a fluorophore, a chromophore, a chemiluminiscent moiety, an enzyme, a radioactive isotope, etc. In some embodiments, the kits comprise a labeled antibody that binds to the lectin.

d. Polycations and Poly-L Lysine

In some embodiments, a poly-L-lysine (or similar polycationic substance) can be used in conjunction with antibodies in a “sandwich” approach as described in herein.

The kit can also contain instructions on how to use a biological sample with the poly-L-lysine (or similar polycationic substance) supplied and the other components necessary to run the desired reaction. The kit further provides instructions that interpret the detectable presence of a DEFB-126 polypeptide as indicative of normal fertility and the absence or reduced presence of a DEFB-126 polypeptide as indicative of an increased risk of infertility. The detected presence of DEFB-126 can also be determined with reference to a predetermined threshold level. In such embodiments, the kit further provides instructions that interpret the presence of a DEFB-126 polypeptide above the threshold level as indicative of normal fertility and the absence or presence of a DEFB-126 polypeptide below the threshold level as indicative of an increased risk of infertility. In some embodiments, the kits contain at least one lectin that selectively binds Galactose-GalNAc or sialic acid. In some embodiments, the kit also contains sperm-specific antibodies.

In some embodiments, poly-L-lysine comprises a detectable label, e.g., with a fluorophore, a chromophore, a chemiluminiscent moiety, an enzyme, a radioactive isotope, etc. In some embodiments, the kits comprise a labeled antibody that binds to the poly-L-lysine (or similar polycationic substance).

e. Polypeptides

In some embodiments, kits of the present invention can also contain a functional DEFB-126 polypeptide for reconstitution of sperm functionality (i.e., the ability to effect conception, e.g., reconstitution of ability for cervical mucus (CM) penetration) to individuals that express mutant or non-functional DEFB-126 polypeptides for use in treating male infertility. In some embodiments, the DEFB-126 polypeptide is obtained from a non-human primate, e.g., a cynomolgus macaque. In some embodiments the DEFB-126 is obtained from humans. In some embodiments the, DEFB-126 can be a recombinant DEFB-126 (e.g., expressed in a eucaryotic expression system such as insect cells). Generally, the kit comprises a functional DEFB-126 polypeptide that allows for reconstitution of normal fertility, as described herein for the therapeutic “add-back” methods and pharmaceutical compositions.

The kit can contain instructions on how to use a biological sample comprising sperm with a functional DEFB-126 polypeptide supplied and the other components necessary to restore sperm functionality (i.e., the ability to effect conception, e.g., reconstitution of ability for cervical mucus (CM) penetration). The kits can further provide instructions and materials for use in interpreting whether the DEFB-126 reconstitution has worked, such as HA or CM penetration gels for analyses. Reconstitution of sperm functionality (i.e., the ability to effect conception, e.g., reconstitution of ability for cervical mucus (CM) penetration) is indicative of normal fertility.

IV. Treatment of Individuals Harboring a DEFB-126 Deletion Polymorphism

a. Replacing a Defective DEFB-126 Polynucleotide

By establishing that an individual carries the DEFB-126 early in an infertility evaluation, clinicians can obtain scientific evidence to justify rapid progression to directed interventions such as intrauterine insemination (IUI) and in vitro fertilization (IVF), thus saving couples the time and expense of a protracted workup.

A wild-type DEFB-126 nucleic acid can be introduced into the epididymis. This can be accomplished by introducing into an epididymal cell of an individual possessing the DEFB-126 deletion polymorphism a nucleic acid encoding a functional DEFB-126 polypeptide. The DEFB-126 nucleic acid sequence can be introduced either in vitro or in vivo. Introduction of a wild-type DEFB-126 gene sequence may be accomplished by any methods of gene therapy known in the art. Any vector known in the art can be used to deliver the therapeutic gene to the epididymis. Viral vectors such as DNA and RNA viral vectors, adenoviruses and adeno-associated viruses may be used as gene therapy vectors. Non-viral vectors such as naked DNA, oligonucleotides, lipoplexes and polyplexes and dendrimers are further examples of methods employed to provide a host cell with recombinant DNA. The gene therapy methods provided above are examples, and the methods disclosed in the present invention should not be limited only to the methods of gene therapy discussed above or currently known methods of gene therapy employed in the art. Therapeutic gene therapy methods are well known in the art (see, e.g., Verma and Weitzman, Ann. Rev. Biochem. 74:711-38 (2005)).

b. Replacing a Defective DEFB-126 Polypeptide

Alternatively or in addition to gene therapy treatment, the wild-type DEFB-126 protein can be used to reconstitute DEFB-126 mutant or deficient sperm, again providing for a less expensive and less invasive therapeutic approach to infertility. The defective or deficient DEFB-126 polypeptide can be replaced either in vivo (e.g., intravaginally) or in vitro. The in vitro functionally restored sperm can be used in in vitro fertilization procedures. The functional DEFB-126 polypeptide can be purified from a natural source (e.g., from a human or non-human primate who produces a functional DEFB-126 protein) or recombinantly produced (e.g., in a eucaryotic expression system such as insect cells). DEFB-126 protein can be purified as described herein and in the art.

The functional DEFB-126 protein can be contacted with sperm from an individual who fails to properly express a DEFB-126 polypeptide. “Adding back” functional DEFB-126 polypeptide to the sperm of an individual who does not express functional DEFB-126 restores DEFB-126 activity. Notably, contacting sperm of an individual who does not express functional DEFB-126 with functional DEFB-126 restores or improves the ability of the DEFB-126-deficient sperm to penetrate the cervical mucus (CM) and effect conception. The soluble wild-type DEFB-126 protein can be used to reconstitute or replace a non-functional DEFB-126 or non-expressed DEFB-126 on the surface of the sperm of an infertile individual. In some embodiments, the soluble wild-type DEFB-126 protein can be used to reconstitute a mutant DEFB-126 protein present on the sperm of an infertile individual. In some embodiments, the soluble wild-type DEFB-126 protein can be used to reconstitute DEFB-126 activity where the DEFB-126 protein is absent from the sperm of an infertile individual.

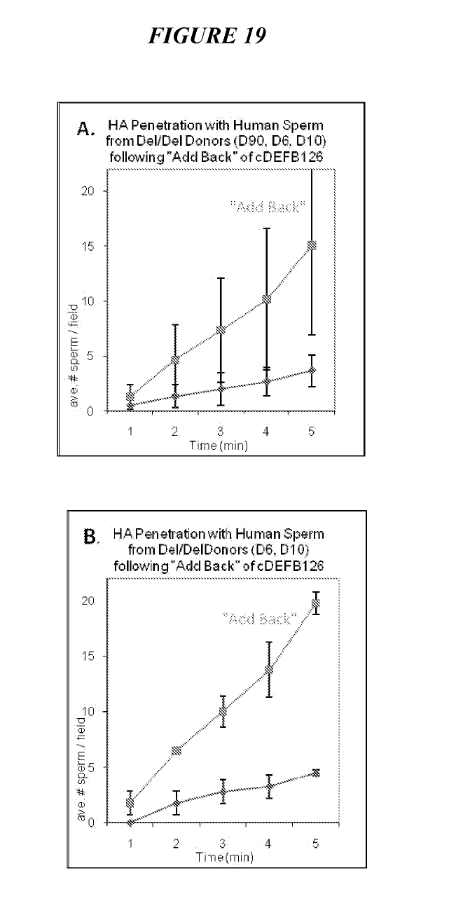

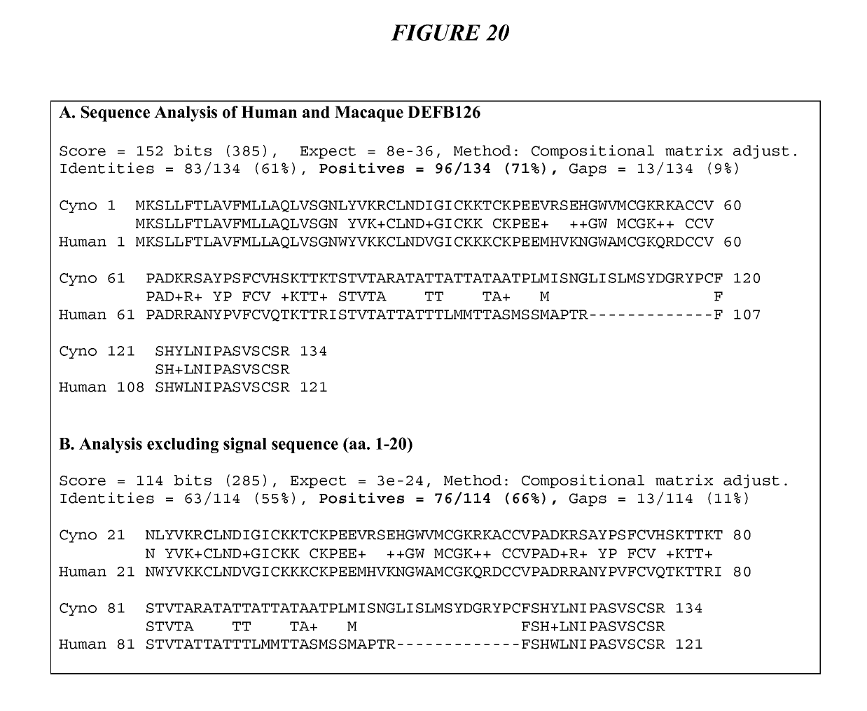

Despite the low amino acid sequence identity (71%; FIG. 20) between DEFB-126 from cynomolgus macaques and humans, the cynomolgus macaque protein can be employed in humans. As shown in FIG. 19, treatment of human male sperm from del/del donor males with wild-type DEFB-126 improved sperm penetration in HA gels. As such, DEFB-126 from cynomolgus macaques has potential for being employed in humans for therapeutic purposes. DEFB-126 obtained from cynomolgus macaques can be employed for fertility treatments in humans. DEFB-126 obtained from cynomolgus macaques can be therapeutically employed in humans in order to reconstitute DEFB-126 activity. In some embodiments, the wild-type soluble DEFB-126 can be from cynomolgus macaques. In some embodiments the defective sperm can be from humans. In some embodiments, therapeutic treatment of infertile human males lacking DEFB-126 or expressing a mutant DEFB-126 can be treated by reconstitution of sperm from wild-type DEFB-126 obtained from cynomolgus macaques.

The DEFB-126 polypeptide or peptide mimetic useful for reconstitution is one that is capable of allowing for normal fertility. Functionally, for methods of reconstituting or restoring the function of DEFB-126 deficient sperm, a functional DEFB-126 polypeptide has two general properties of the native DEFB126 molecule: (1) the ability to bind reversibly to the sperm surface depending on sperm capacitation state, and (2) the ability to impart a negative charge to the sperm surface while bound. The first property of DEFB126 (and its orthologs) is mediated by the beta-defensin “core” or motif (e.g., SEQ ID NOs: 46-49, discussed above). While beta-defensins can differ considerably in amino acid sequence, the position of 6 cysteine residues are strictly conserved and induce via disulfide bonds a characteristic folding of the peptide. Beta-defensins are also highly cationic, due to an excess of arginine and lysine residues relative to the number of aspartate and glutamate residues. Beta-defensins also have a high proportion of hydrophobic amino acid residues. The combination of the 3-dimensional structure, high positive charge, and high proportion of hydrophobic residues enables beta-defensins to bind to membrane surfaces. The second property of DEFB126 is imparted by the negatively charged carboxyl extension of the peptide. In the native molecule, sialylated O-linked oligosaccharides accounts for most of the charge, but alternative structures, such as N-linked oligosaccharides, can render the carboxyl end of the polypeptide sufficiently anionic. O-linked carbohydrates are bound to serine and threonine, whereas N-linked carbohydrates are bound to asparagine.

Accordingly, in some embodiments, the DEFB-126 polypeptide or polypeptide mimetic comprises a core beta-defensin motif (aa 21-67), e.g., a polypeptide comprising an amino acid sequence having 95%, 96%, 97%, 98%, 99% or 100% sequence identity to SEQ ID NO:46, SEQ ID NO:47, SEQ ID NO:48, SEQ ID NO:48 or SEQ ID NO:49. Where there are no shared residues among the orthologs (−) (aligned above), amino acids can be substituted that are similar in charge or polarity or that contribute to retention of charge and polarity of inter-cysteine spans.

In some embodiments, the DEFB-126 polypeptide or polypeptide mimetic comprises a carboxyl extension motif (e.g., aa 68-121, 68-134; or 68-181) that is sufficiently anionic to impart a negative charge to the sperm surface while bound, e.g., has a sufficient number of N-linked carbohydrates, e.g., sialic acid moieties. The carboxyl extension of the DEFB126 orthologs varies considerably in both sequence and length. Similarities include an abundance of serine and threonine residues (˜40% of total residues in carboxyl region) along with proline, and arginine residues that make glcosylation at neighboring sereine and threonine residues more likely. All orthologs also contain a seventh cysteine that proceeds the glycosylated region of the carboxyl extension (shown in brackets). It is not clear if the bracketed region contributes to the general properties of the native molecule and therefore this sequence segment can optionally be included or deleted in a functional DEFB-126 polypeptide, as desired. Below is COOH-end structure based on the mouse sequence and is the longest of the orthologs. Shorter structures (based on the cyno and human DEFB-126 sequences) will also be functional, e.g., aa68-121, aa68-134 or aa68-181, provided they are sufficiently anionic to impart a negative charge to the sperm surface while bound, e.g., have a sufficient number of N-linked carbohydrates, e.g., sialic acid moieties.

(SEQ ID NO: 50)

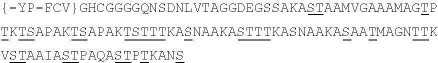

In some embodiments, the functional DEFB-126 polypeptide or polypeptide mimetic comprises a defensin core motif and a defensin carboxyl extension motif, e.g., from the same or a different species. In some embodiments, the functional DEFB-126 polypeptide or polypeptide mimetic comprises a defensin core motif and a carboxy motif that comprises one or more tandem repeats or sequence segments that allow for O-linked and/or N-linked glycosylation (e.g., mucin repeat sequences) such that the polypeptide is sufficiently anionic to impart a negative charge to the sperm surface while bound. In some embodiments, the functional DEFB-126 polypeptide or polypeptide mimetic comprises a defensin core motif of SEQ ID NOs: 46, 47, 48 or 49 and a defensin carboxyl extension motif of SEQ ID NO:50, or shorter lengths of SEQ ID NO:50 (e.g., aa 68-121, 68-134; or 68-181) with sufficient anionic charge to impart a negative charge to the sperm surface while bound.

In some embodiments, the DEFB-126 polypeptide that allows for normal fertility shares at least 95%, 96%, 97%, 98%, 99% sequence identity to a wild-type DEFB-126 polypeptide selected from SEQ ID NO:6, SEQ ID NO: 7, SEQ ID NO:8, SEQ ID NO:9, SEQ ID NO:10, SEQ ID NO:11 and SEQ ID NO:12. In some embodiments, the DEFB-126 polypeptide that allows for normal fertility shares at least 95%, 96%, 97%, 98%, 99% sequence identity to a wild-type DEFB-126 polypeptide of SEQ ID NO:6. In some embodiments, the DEFB-126 polypeptide that allows for normal fertility shares at least 95%, 96%, 97%, 98%, 99% sequence identity to a wild-type DEFB-126 polypeptide of SEQ ID NO:12.

Functional DEFB-126 polypeptides and polypeptide mimetics that find use in the present compositions and methods will restore the function of a DEFB-126 deficient sperm sample to penetrate a CM or HA gel, and can be tested in the HA/CM gel penetration assays, described herein.

### DEFB-126 “Add-Back” Assays

a. Preparation of Soluble DEFB-126

Sperm that lack DEFB-126 or contain a mutant are defective for cervical mucus (CM) penetration, resulting in infertility. DEFB-126 can be added back to reconstitute sperm functionality (i.e., the ability to effect conception, e.g., reconstitution of ability for cervical mucus (CM) penetration) to DEFB-126 defective or deficient sperm and potentially render infertile males fertile.

DEFB-126 soluble protein can be obtained by any methods well known to one of skill in the art. General methods for protein preparation have been well described (see generally, Sambrook et al. Molecular Cloning: A Laboratory Manual, 3rd ed., Cold Spring Harbor Laboratory Press, Cold Spring Harbor, N.Y. (2001) and Ausubel, ed., Current Protocols in Molecular Biology, John Wiley Interscience, (1990-2008)).

Specific methods for preparation of a solution containing soluble DEFB-126 have been well described (Tollner et al., (2004)). Briefly sperm samples were washed through a 3.5-ml column of 80% Percoll, washed, resuspended and the DEFB-126 protein concentration determined, as described in Tollner et al., (2004), and resuspended in 10 ml DPBS without energy substrates and BSA.

b. Add-Back/Reconstitution Assays

Protein add-back experiments are common and well known in the art and have been well described (Tollner et al., (2004, 2008a,b)). DEFB-126 can be added to sperm deficient for DEFB-126 as well as to sperm with a mutant or non-functional DEFB-126. DEFB-126 can be absent from sperm, due to for example the presence of a non-expressing allele in an individual. DEFB-126 can be removed from sperm, e.g., using ACT, which is known in the art to remove DEFB-126 from the sperm surface (Tollner et al., (2004)).

Interestingly, despite the fact that the DEFB-126 from cynomolgus macaques is only 71% homologous to the human protein (FIG. 20), reconstitution of human sperm using the DEFB-126 from the cynomolgus macaques was effective in improving human sperm functionality (Example 9 and FIG. 19). In some embodiments, the wild-type soluble DEFB-126 from macaques or another non-human primate can be used for add-back experiments in order to reconstitute human sperm function.

c. Cervical Mucus or HA Penetration Gels

Cervical mucus (CM) gels can be prepared by collection of cervical mucus from peri-ovulatory female cynomolgus macaques. After collection, CM can be formulated onto a microscope slide for use in sperm motility experiments. These experiments have been well described in the art (Tollner et al., (2008b)).

Medium composed of hyaluronic acid (HA) has been used to simulate CM for in vitro tests of sperm function. While CM has complex biophysical properties that are derived from at least five distinct mucin molecules produced at the cervix (Gipson et al., 1997; Lagow et al., 1999), solutions prepared from HA share some of the properties of mucus, especially with respect to viscosity and charge (Gatej et al., 2005). HA gels highly resemble CM in their penetrability by human sperm (Tang et al., 1999; Neuwinger et al., 1991; Aitken et al., 1992). Due to the limited availability and high variability of human CM, HA gels have been used as mucus surrogates in clinical assessment of sperm function (Aitken, 2006). Video analysis can be performed to examine the movement of individual sperm in the HA penetration gel. (see, e.g. Tollner et al., (2008) and Cherr et al., (1999)).

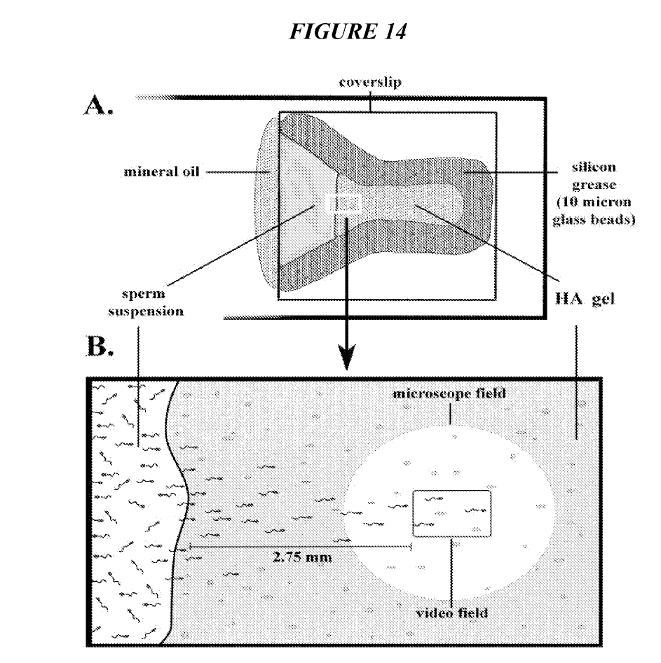

CM or HA gels can be formulated into a chamber for analysis. These chambers allow for videomicrographic analysis of sperm penetration ability and have been described in the art (Tollner et al 2008b). A general diagram of a penetration chamber containing a CM or HA gel is shown in FIG. 14.

Penetration chambers can be employed for analysis of the penetration ability of sperm obtained directly from an individual, sperm pretreated to remove proteins, or sperm to which proteins have been added back. In some embodiments, sperm can contain a mutant DEFB-126. In some embodiments sperm can be pretreated to remove the DEFB-126. In some embodiments, sperm can be pre-treated to remove DEFB-126 and then DEFB-126 added back.

Efficient sperm movement in CM has been described as important for fertility. In fact, removal of DEFB-126 has been shown to decrease the ability of sperm to penetrate (or move efficiently in) in cervical mucus (CM). The CM and HA penetration chambers described above can additionally be employed for diagnostic purposes for examining the function of sperm obtained directly from an individual. (see, e.g., Tollner, et al., 2008b, as well as Example 8 and Table 2).

V. Compositions Comprising a Functional DEFB-126 Polypeptide

The invention further provides compositions comprising a functional DEFB-126 polypeptide in a physiologically acceptable carrier. In one embodiment, the pharmaceutical compositions comprises a functional non-human DEFB-126 polypeptide or DEFB-126 polypeptide mimetic, as described herein, for use in restoring or improving the sperm functionality of a human sperm expressing insufficient DEFB-126 to effect conception.

The DEFB-126 polypeptide or peptide mimetic useful in the present compositions is one that is capable of allowing for normal fertility. A functional DEFB-126 polypeptide has two general properties of the native DEFB126 molecule: (1) the ability to bind reversibly to the sperm surface depending on sperm capacitation state, and (2) the ability to impart a negative charge to the sperm surface while bound.

Accordingly, in some embodiments, the DEFB-126 polypeptide or polypeptide mimetic in the present compositions comprises a core beta-defensin motif (aa 21-67), e.g., a polypeptide comprising an amino acid sequence having 95%, 96%, 97%, 98%, 99% or 100% sequence identity to SEQ ID NO:46, SEQ ID NO:47, SEQ ID NO:48, SEQ ID NO:48 or SEQ ID NO:49. Where there are no shared residues among the orthologs (−) (aligned above), amino acids can be substituted that are similar in charge or polarity or that contribute to retention of charge and polarity of inter-cysteine spans.

In some embodiments, the DEFB-126 polypeptide or polypeptide mimetic comprises a carboxyl extension motif (e.g., aa 68-121, 68-134; or 68-181) that is sufficiently anionic to impart a negative charge to the sperm surface while bound, e.g., has a sufficient number of N-linked carbohydrates, e.g., sialic acid moieties.

In some embodiments, the functional DEFB-126 polypeptide or polypeptide mimetic comprises a defensin core motif and a defensin carboxyl extension motif. In some embodiments, the functional DEFB-126 polypeptide or polypeptide mimetic comprises a defensin core motif and a carboxy motif that comprises one or more tandem repeats or sequence segments that allow for O-linked and/or N-linked glycosylation (e.g., mucin repeat sequences) such that the polypeptide is sufficiently anionic to impart a negative charge to the sperm surface while bound. In some embodiments, the functional DEFB-126 polypeptide or polypeptide mimetic comprises a defensin core motif of SEQ ID NOs: 46, 47, 48 or 49 and a defensin carboxyl extension motif of SEQ ID NO:50, or shorter lengths of SEQ ID NO:50 (e.g., aa 68-121, 68-134; or 68-181) with sufficient anionic charge to impart a negative charge to the sperm surface while bound.

In one embodiment, the functional DEFB-126 polypeptide is prepared in pharmaceutical compositions formulated for topical administration, for instance, in a cream, a paste, a gel, a foam, an ointment, a spray, a lubricant, an emulsion or suspension. In some embodiments, the pharmaceutical compositions formulated for topical administration comprise a functional DEFB-126 polypeptide that is at least 95% identical to an amino acid sequence selected from SEQ ID NO:6, SEQ ID NO:7, SEQ ID NO:8, SEQ ID NO:9, SEQ ID NO:10, SEQ ID NO:11 and SEQ ID NO:12.

In topical formulations, usually the functional DEFB-126 polypeptide is included in about 0.1, 0.2, 0.5, 1.0 or 2.0 wt %, but can be included in as much as 5, 10, 15 or 20 wt % of the total formulation, or more. The functional DEFB-126 polypeptide is formulated with one or more pharmaceutically acceptable carriers. For topical applications, the pharmaceutically acceptable carrier may additionally comprise organic solvents, emulsifiers, gelling agents, moisturizers, stabilizers, other surfactants, wetting agents, preservatives, time release agents, and minor amounts of humectants, sequestering agents, dyes, perfumes, and other components commonly employed in pharmaceutical compositions for topical administration. Solid dosage forms for topical administration include suppositories, powders, and granules. In solid dosage forms, the compositions may be admixed with at least one inert diluent such as sucrose, lactose, or starch, and may additionally comprise lubricating agents, buffering agents and other components well known to those skilled in the art.

Functional DEFB-126 polypeptide formulations suitable for vaginal administration can be presented as pessaries, tampons, creams, gels, pastes, foams, or spray formulas.

### EXAMPLES

The following examples are offered to illustrate, but not to limit the claimed invention.

### Example 1

### DEFB-126 Deletion Polymorphism has a Significantly Altered Peptide Structure

While cloning human DEFB-126 for recombinant expression in a prokaryotic system, a sequence variation in DEFB-126 cDNA was identified. This specific DEFB-126 deletion polymorphism appears to be very common in the human population. The amino acid sequence of the variant DEFB-126 has a significantly altered and extended carboxyl terminal, carbohydrate-containing domain which causes a profound alteration in structure and function. Furthermore, an epididymal specimen with the sequence variant has markedly lower expression of DEFB-126 compared to wild-type epididymal tissue.

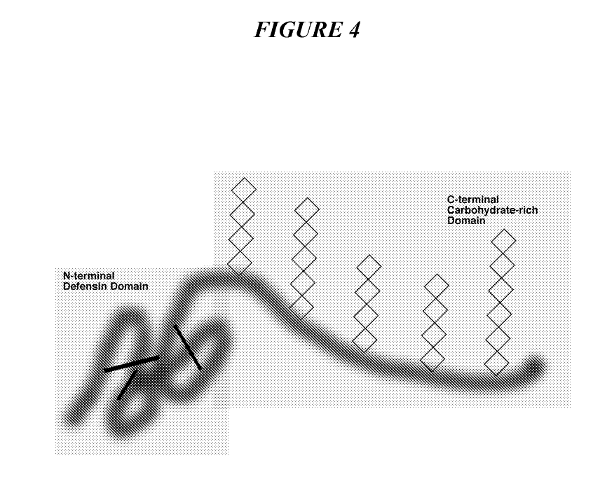

The DEFB-126 variant had a 2-nucleotide omission (deletion), causing a frame-shift in the open reading frame of DEFB-126 (FIG. 4A). This sequence variation was confirmed in the NCBI genomic DNA sequence database. 465 randomly selected individuals from a cohort of Chinese men were genotyped (collaboration with S. Venners & Xiping Xu, U. Chicago) in addition to 74 individuals from a population of men in Great Britain (FIG. 4B). mRNA encoding aberrant protein is often present in lower steady state concentrations because of more rapid degradation. Therefore, DEFB-126 mRNA was analyzed in epididymal tissue using quantitative RT-PCR (qPCR). A reduced level of DEFB-126 mRNA was analyzed in an epididymal specimen with the sequence variant (FIG. 4C). Furthermore, the amino acid sequence of the variant DEFB-126 has a significantly altered carboxyl terminal, carbohydrate-containing domain (FIG. 4D).

### Example 2

### DEFB-126 Deletion Polymorphism in Men Associated with Reduced Fertility

The DEFB-126 deletion polymorphism was genotyped in 638 men who took part with their wives in a population-based, prospective, cohort study of fertility and pregnancy in agricultural communities in Anhui Province, China. All had been recently married at recruitment between July, 2003 and February, 2005 and none were pregnant at enrollment. Couples were excluded from this analysis for female-related factors of infertility including menstrual irregularity and pelvic inflammation (n=81) and in a subset analysis, for male factors including evidence of chronic bacterial prostatitis (semen pH>8.0; n=110). Fifty-nine couples were excluded who had used oral contraceptives or IUD within the year prior to enrollment. Data were missing for another 39 couples who were lost to follow-up. This analysis includes the remaining 355 couples.

The data were analyzed by logistic regression for the relative odds of pregnancy within 21 months according to the husband's DEFB-126 genotype. The analysis showed that men with the del/del genotype were significantly less fertile (OR=0.5, p=0.03) Table 1. The model allowed independent parameters within levels of semen pH for the associations between DEFB-126 genotype and pregnancy, but all other parameters in the model were estimated across the entire population. The logistic regression parameters were adjusted for husbands' age, wives' body-mass index and husbands' days of sexual abstinence prior to semen collection. None of the husbands' body-mass index or smoking or wives' age were statistically significant in this model as α=0.20. When the data for sperm count (>20 million sperm/ml or <20 million sperm/ml; World Health Organization Reference Value) were analyzed using the same model there was no association with pregnancy within 21 months. The DEFB-126 genotype was associated with fertility in this prospective cohort, while sperm count, a traditional measure of male fertility, was not.

TABLE 1
Logistical Regression Parameters for Relative Odds of Pregnancy

| | | | Crude | | Adjusted * | |
|---|---|---|---|---|---|---|
| | Pregnancies | | OR | | OR | |
| | n | n (%) | (95% CI) | p | (95% CI) | p |
| DEFB-126 | | | | | | |
| WT/WT-WT/ | 285 | 231 (81%) | Ref | | Ref | |
| Del | | | | | | |
| Deletion/ | 70 | 48 (69%) | 0.5 | .026 | 0.5 | .030 |
| Deletion | | | (0.3, 0.9) | | (0.3, 0.9) | |
| Sperm Count | | | | | | |
| ≧20 × 10⁶ | 274 | 216 (79%) | Ref | | Ref | |
| sperm/ml | | | | | | |
| <20 × 10⁶ | 191 | 151 (79%) | 1.0 | .882 | 0.9 | .621 |
| sperm/ml | | | (0.6, 1.5) | | (0.5, 1.5) | |

* Adjusted for husbands' age, wives' body-mass index and husbands' days of sexual abstinence prior to semen collection. None of husbands' body-mass index or smoking or wives' age were statistically significant in this model at α = 0.20.

### Example 3

### Sperm from Donors Possessing the DEFB-126 Deletion Polymorphism Have Reduced Surface Glycosylation Associated with O-Linkages

Human sperm from donors possessing wt and variant genotypes were labeled with the lectin Agaricus bisporus (ABA) which selectively binds Galactose-GalNAc-serine (or threonine), structures specific of O-linked glycans. Sperm from donors possessing the DEFB 126 variant (del/del) showed significant reduction in ABA-associated fluorescence compared to wild type donors (wt/wt; wt/del). Amino acid sequence analysis of human DEFB126 identifies at least 20 sites for O-linked glycosylation. The significant reduction in binding sites for ABA suggests that DEFB 126 is either not adsorbed to the surface of sperm from men possessing the DEFB-126 deletion polymorphism or lacks most, if not all of its oligosaccharides.

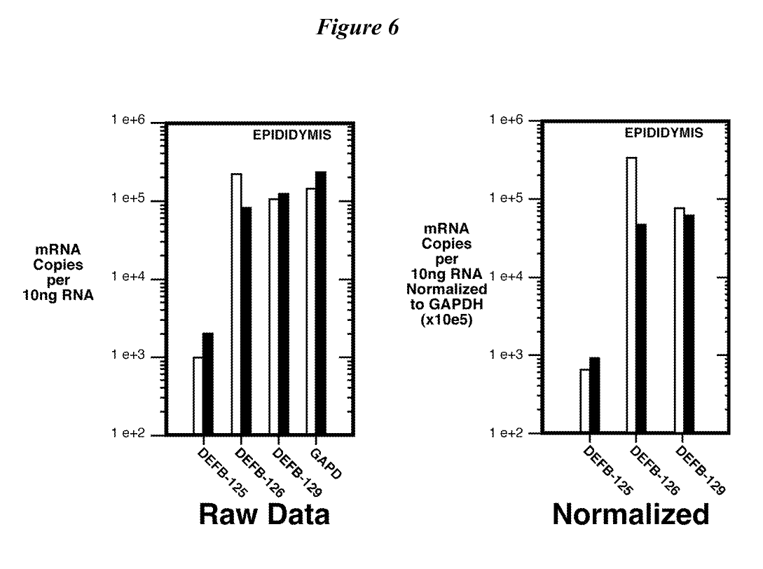

Human sperm were treated with neuraminidase, fixed with paraformaldehyde/gluteraldehyde, and incubated with FITC-conjugated lectin ABA. Differences in fluorescence can clearly be seen between sperm from DEFB126 wild type donors (wt/wt and wt/del) and sperm from donors that carry only the DEFB 126 gene variant (del/del; FIG. 6A). Intensity of lectin-labeled sperm from the same donor was quantitated with MetaMorph Image Analysis software. Digital images of 36 sperm/donor were thresholded to the same level for all treatments and average pixel intensity was determined (FIG. 6B).

### Example 4

### Fine-Structure Characterization of Sperm Surface Oligosaccharides

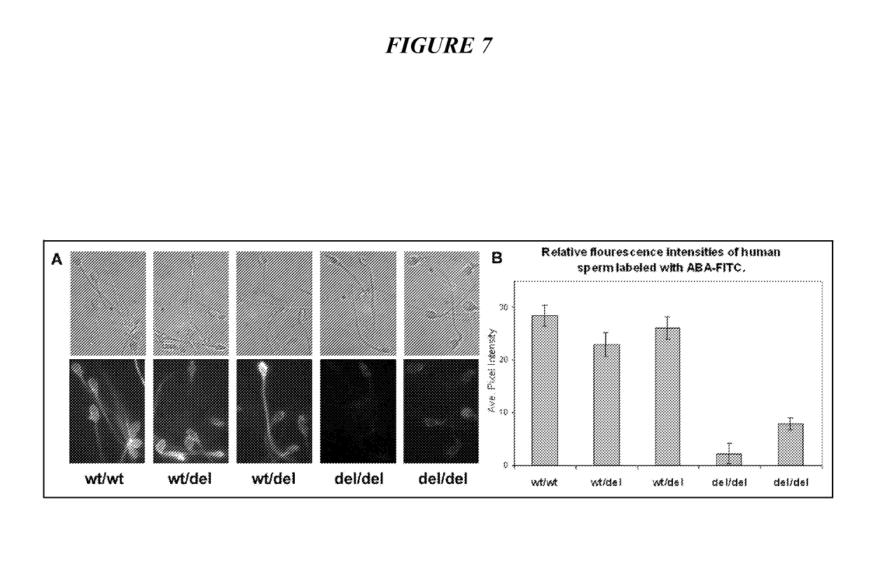

The mass spectrum of O-linked oligosaccharides excised from purified DEFB 126 suggests that the oligomers are highly sialylated, a finding that is consistent with the highly anionic nature of the glycoprotein. Human sperm were treated with neuraminidase, fixed with paraformaldehyde/glutaraldehyde, and incubated with FITC-conjugated lectin ABA. Differences in fluorescence can clearly be seen between sperm from DEFB 126 wild type donors (wt/wt and wt/del) and sperm from donors that carry only the DEFB 126 gene variant (del/del; FIG. 7A). Intensity of lectin-labeled sperm from the same donor was quantified with MetaMorph Image Analysis software. Digital images of 36 sperm/donor were thresholded to the same level for all treatments and average pixel intensity was determined (FIG. 7B). There was insufficient amount of oligosaccharides to perform tandem MS for the preliminary results. However, more oligosaccharides will be obtained in subsequent studies proposed here that will allow structural elucidation and monosaccharide composition analysis with tandem MS.

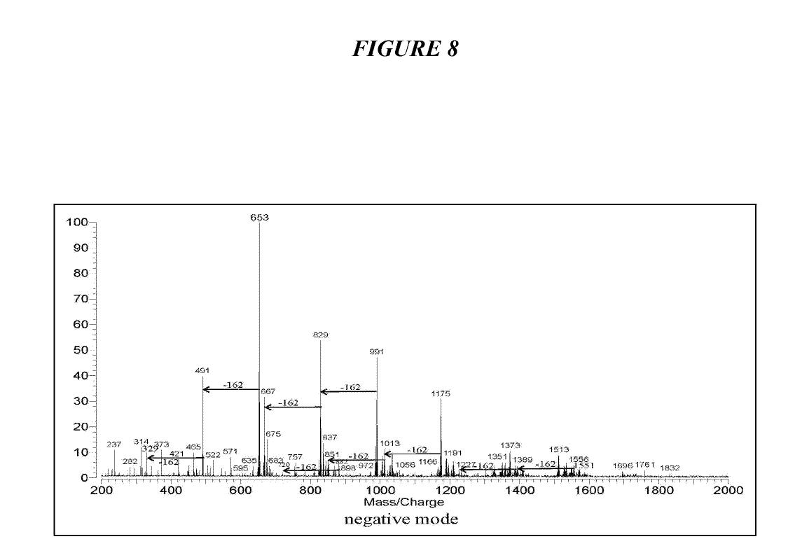

The relative small size of the protein makes it amenable for so called “top-down” analysis where the intact glycoprotein, or the major glycosylated tryptic peptide can be probed directly with mass spectrometry. Shown in FIG. 8A is the mass spectrum of another defensin (Lebrilla, Bevins, et al., unpublished) peptide expressed as the deglycosylated form. The same defensin peptide obtained with its constituent glycan intact shows the heterogeneity of the glycan structures as a series of peaks differing by distinct monosaccharide masses. See FIG. 8C, inset deconvoluted spectrum. By probing the intact glycopeptide with tandem MS, we can obtain both the glycan constituent and the site specific glycosylation.

Oligosaccharides were released from DEFB-126 and examined with MS. To obtain samples for analyses the purified protein was treated with NaBH₄/NaOH to release O-linked oligosaccharides. For clean-up, the sample was passed through a porous graphitic carbon cartridge to collect and separate the oligosaccharides. The mass spectrum obtained on the MALDI FTMS instrument shows a number of characteristics corresponding to oligosaccharides. The peaks 162 mass units apart correspond to oligomers with hexose residues. The groups of peaks, for example between 990 and 1056 indicate the presence of oligomers containing sialic acids. The species is distributed over several signals containing a number of Na⁺ ions.

### Example 5

### DEFB-126 is Secreted into the Epididymal Duct and is Adsorbed Onto the Sperm Surface

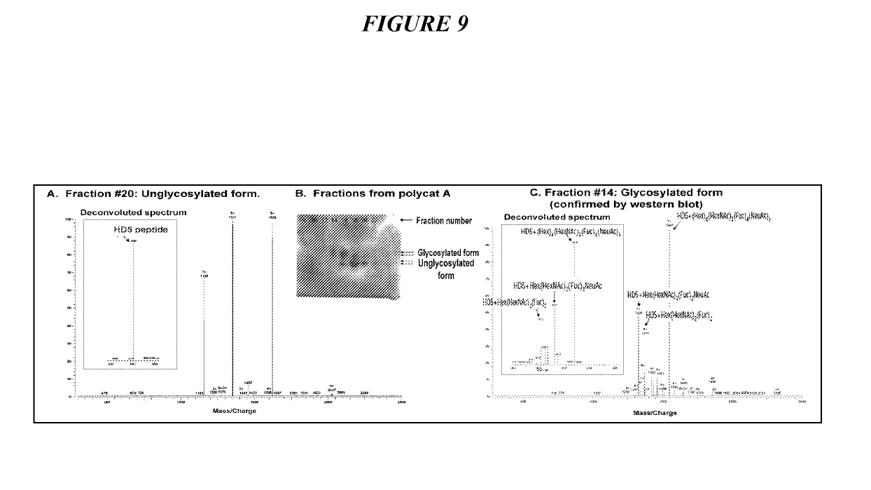

Expression of DEFB-126 in the corpus epididymis appears to be a conserved feature of mammalian sperm maturation. Expression of DEFB-126 in the distal corpus has been described previously in the macaque (Perry, et al., Biol. Reprod. 61:965-972 (1999)) and in the human (Rodriguez-Jimenez et al., Genomics 81:175-83 (2003)). Corpus expression of DEFB126 was verified in the macaque (FIG. 9A; Yudin et al., Biol. Reprod. 69:1118-1128 (2003)). Corpus expression of the DEFB-126 ortholog (Defb22) was verified in the mouse (FIGS. 9B and C). DEFB 126 (and mouse ortholog Defb22) becomes adsorbed over the entire surface of sperm in transit to the cauda.

Antibodies to DEFB-126 detect on western blots of male macaque reproductive tissues a heavily glycosylated DEFB-126 migrating at 31-36 kDa (FIG. 9A). Antibodies to the mouse ortholog of DEFB126 similarly detected a glycopeptide in the mouse epididymis

(FIG. 9B). In both the monkey and mouse, expression of the glycosylated defensin (DEFB126/Defb22, respectively) appears to start in the corpus and the defensin remains associated with sperm. Defb22 antigen was localized on paraffin sections of the caput and corpus regions of the mouse epididymis (FIG. 9C). The various segmented regions were apparent when stained with Papanicolaou (“PAP”; E). In the proximal corpus, the lumen of the epididymis failed to stain with the antibody, but the peripheral edge of the duct was heavily labeled with the anti-Defb22 Ig. As the sperm progress to the distal portion of the corpus the lumen becomes filled with sperm and those sperm were heavily labeled with anti-Defb22 Ig (FIG. 9C, lower right). Antibodies specific to DEFB126 and Defb22 recognize the glycosylated defensin on washed caudal macaque and mouse sperm, respectively. In both species, the defensin is distributed over the entire surface of sperm.

### Example 6

### The DEFB-126 Surface Coat Facilitates Various Phases of Sperm Transport in the Female Tract

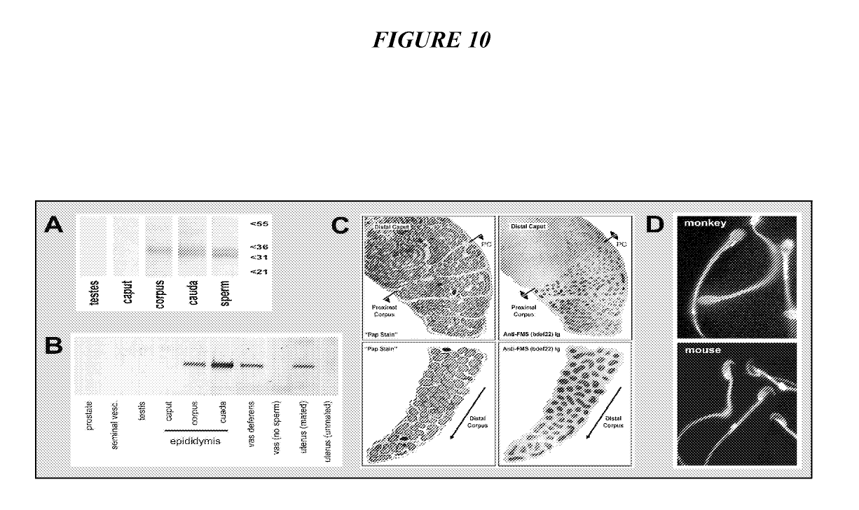

DEFB-126 is retained on macaque sperm recovered from the upper reproductive tract of mated female macaques (Tollner et al., Hum. Reprod. 23:2523-34 (2008)). DEFB-126 on the macaque sperm surface appears to be critical for a number of key events during sperm transport to this site. Macaque sperm treated with anti-DEFB-126 Igs exhibited greatly reduced ability to penetrate peri-ovulatory cervical mucus (FIG. 10A). The relative magnitude of inhibition was similar to when sperm were treated to remove DEFB-126 (FIG. 9B; Yudin et al., Biol. Reprod. 69:1118-28 (2003)). “Add-Back” of DEFB126 completely restores mucus penetration ability of macaque sperm (FIG. 10B). Treatment of sperm with neuraminidase (which removes terminal sialic acid residues) sharply reduces the net negative charge of DEFB 126 (Yudin et al., Biol. Reprod. 73:1243-1252 (2005)) and significantly inhibits mucus penetration FIG. 10C. Similarly, when the negative surface charge of macaque sperm is neutralized by the addition of poly-L-lysine mucus penetration is inhibited. FIG. 10D. Furthermore, the loss of DEFB-126 exposes numerous sperm-specific surface proteins to antibody recognition (Yudin et al., Biol. Reprod. 73(6):1243-52 (2005)) and decreases the affinity of sperm binding to oviductal epithelia (Tollner et al., Biol. Reprod. 78:400-412 (2008)). Yet the loss of DEFB 126 from the head of sperm is essential in order for sperm to bind to the zona pellucida (Tollner et al., Mol. Reprod. Dev. 69:325-37 (2004)).

Sperm were treated with antibodies specific to DEFB-126 (FIG. 10A), with activator compounds (“released”) to remove DEFB-126 (FIG. 10B), with neuraminidase (FIG. 10C) or with poly-L-lysine (PL, D). Following treatments, sperm were deposited into slide chambers containing peri-ovulatory CM. After 2 minutes, sperm were recorded continuously for 4 minutes as they entered a video field 2.75mm from the sperm suspension-CM interface. Numbers of sperm in the video field were counted at one-minute intervals. For the experiment demonstrated in FIG. 10B, following removal from activator conditions, an aliquot of these sperm were treated with DEFB-126 (Add-Back). Experiments were conducted with sperm from 3-4 different male macaques. Letters (a,b) indicate significant differences (p<0.05) in mean sperm numbers between treatments within time intervals.

### Example 7

### Sperm from Del/Del Donors Exhibit Reduced Ability in Penetrating Gels that Simulate Periovulatory Cervical Mucus (CM)

Medium composed of HA has been used to simulate CM for in vitro tests of sperm function. While CM has complex biophysical properties that are derived from at least five distinct mucin molecules produced at the cervix (Gipson et al., 1997; Lagow et al., 1999), solutions prepared from HA share some of the properties of mucus, especially with respect to viscosity and charge (Gatej et al., (2005)). HA gels highly resemble CM in their penetrability by human sperm (Tang et al., (1999); Neuwinger et al., (1991); Aitken et al., (1992)). Due to the limited availability and high variability of human CM, HA gels have been used as mucus surrogates in clinical assessment of sperm function (Aitken, (2006)).

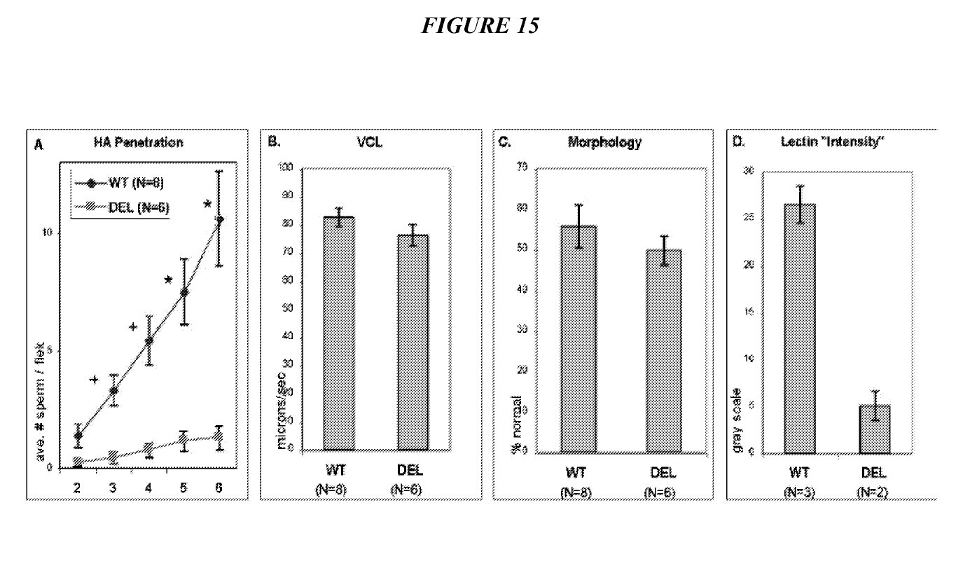

Evaluation of the ability of human sperm to penetrate a hyaluronic acid (HA) gel is shown in FIGS. 14 and 15. FIG. 14 provides a diagram of the sperm penetration of Cervical Mucus (CM) or HA gels. Penetration chambers containing either CM or HA gel were prewarmed on a microscope stage warmer and pre-cued with videomicrographic equipment as described by Tollner et al. (2008b). Sperm were washed into HEPES-buffered sperm medium and deposited into slide chambers. After 2 minutes, sperm were recorded continuously for 4 minutes as they entered a video field 2.75mm from the sperm suspension-CM (or HA gel) interface. From video recordings, the numbers of sperm in the video field were counted at one-minute intervals.

FIG. 15 provides data describing human sperm penetration of HA gel. Sperm from donors genotyped for the DEFB 126 polymorphism were used in HA penetration experiments (A). HA gel was composed of 5mg purified hyaluronate (220 kDa fraction) per ml of HEPES-buffered BWW medium supplemented with 3% BSA. As in fertility cohort study, outcomes for sperm from wt/wt and wt/del donors were averaged together (wt; n=8) and compared with the average response of sperm from del/del donors (del; n=6). Sperm suspensions were analyzed by CASA for average curvilinear velocity (VCL) (B). Slides with smears of sperm suspensions were “Pap”-stained and analyzed according to WHO ‘89 sperm morphology method and reported as total average percent normal forms (% normal) (C). Observations reported in A-C were paired and represent data averaged across two to three ejaculates (sub-samples) from each donor. Data was reported as means +/−SEM. Crosses (+) and asterisks (*) indicate significant differences at p<0.01 and p<0.005, respectively in mean sperm numbers between genotypes as determined by 1-way ANOVA. ABA lectin labeling outcomes were averaged across sperm from donors possessing wt DEFB 126 gene (wt=wt/wt+wt/del) and sperm from donors that possessed only the gene variant (del/del=del) (D). Lectin studies have been extended to included the more recently recruited donors (FIG. 2 of supplement) and will be analyzed for label intensity with Metamorph as described for the original data set.

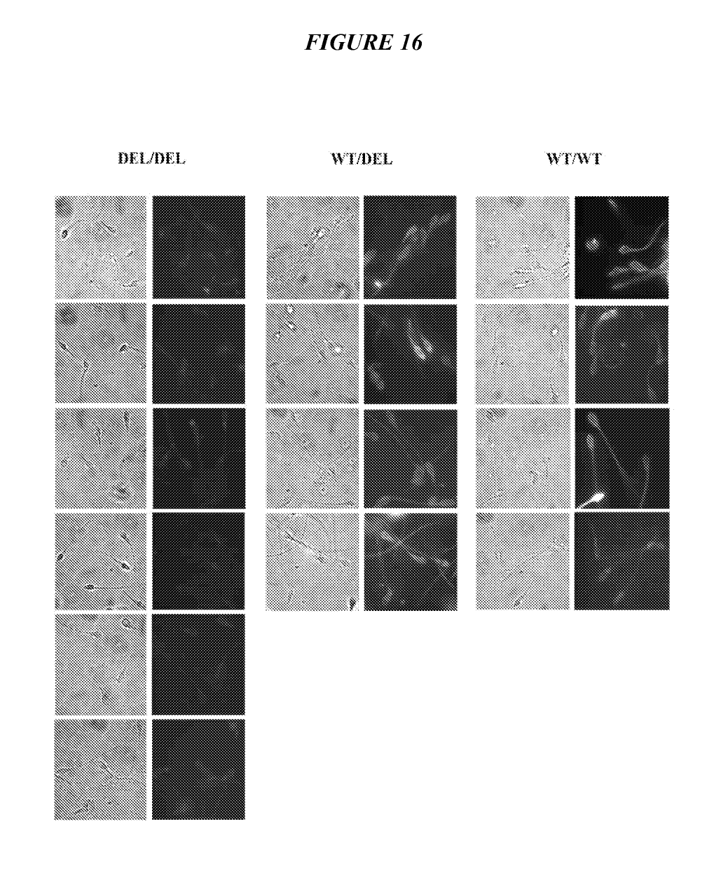

Human sperm from same 14 donors genotyped for the DEFB126 gene variant were tested for the ability to penetrate HA gels (FIG. 15). Sperm from donors that were homozygous for the DEFB 126 polymorphism (del/del=“del”) exhibited significantly reduced HA penetration ability compared to sperm from men possessing at least one copy of the wild type (wt/wt and wt/del=“wt”) (FIG. 15A). Progressive motility, as estimated by average curvilinear velocity (VCL), and morphology of sperm used in penetration assays did not differ significantly between del and wt males and therefore appeared to be poor predictors of sperm performance in HA (FIGS. 15B and 15C). By contrast, DEFB126 genotype and lectin labeling (FIG. 15D) strongly coincided with results of the HA penetration assay. Sperm of all 6 del donors have markedly reduced surface labeling with Agaricus bisporus (ABA) lectin compared to sperm from the 8 wt donors, suggesting that the DEFB126 genetic polymorphism results in a loss or reduction 0-linked oligosaccharides in the sperm glycocalyx (FIG. 16).

### Example 8

### Fertility of Donors is More Accurately Predicted by Variant Genotyping and Sperm Lectin Labeling Than by WHO Normal Semen Reference Values

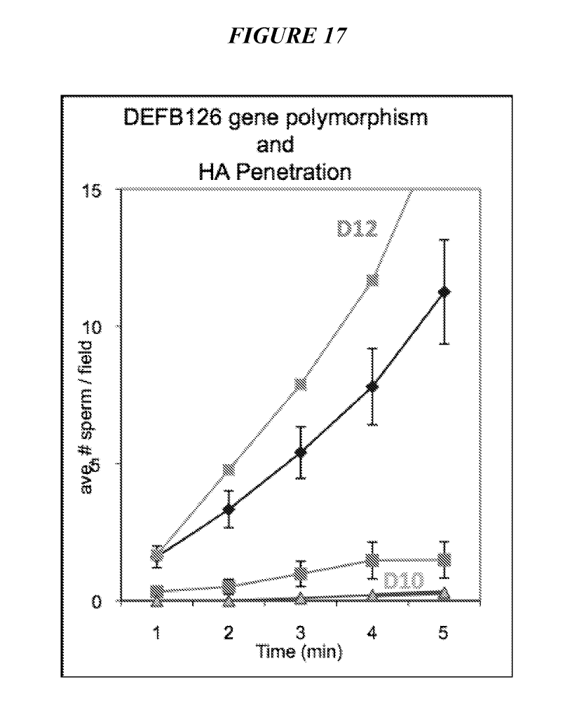

Of the 14 donors we genotyped for lectin and HA penetration studies, 10 are students who have never attempted to conceive with a partner nor have unintentionally achieved conception. Three donors (two wt/del and 1 wt/wt) have fathered children through natural means and one donor (del/del) has fathered a child following clinical interventions at an Assisted Reproductive Technologies (ART) program. WHO reference values for normal semen parameters (Table 2) indicate that donor #10 (D10; del/del) has normal values and would be classified as fertile. In actuality D10 is infertile. He and his spouse (neither of which had any detectable fertility issues at time of clinical work up) attempted to conceive for several years and only conceived after 6 cycles of intrauterine insemination (IUI). By contrast, donor #12 (D12; wt/wt) has sub par sperm morphology and a very low sperm count. By WHO standards he would be classified as most likely infertile or subfertile. D12 and his partner conceived spontaneously during a lapse in the use of birth control. Table 2 highlights circumstances where commonly accepted diagnostic standards fail to predict infertility. In cases of both D10 and D12, results of genotyping and lectin labeling (FIG. 16) strongly agree with tests of sperm function (FIG. 17) and actual fertility status.

TABLE 2
WHO “Normal” Semen Values

| Parameters | WHO | D10 | D12 |
|---|---|---|---|
| Morphology | 50% | 49.8 ± 7.8 | 38.7 ± 2.1 |
| (% Normal Forms) | | | |
| % Progressive Motility | 50% | 54.3 ± 4.7 | 56 ± 13.9 |
| Sperm Density. (millions/ml) | 20 | 59 ± 16.6 | 7.1 ± 3.5 |
| Total Sperm/ejac. (millions) | 40 | 241.9 | 24.9 |
| WHO Assessment: | | Fertile | Subfertile |
| Actual Fertility Status: | | Infertile* | Fertile* |

*Ideopathic infertility: 3 years of attempting to conceive by natural means, including 18 months with ovulation monitering. Conceived after 6 cycles of IUI.
*Spontaneous Pregnancy: unplanned conception occurred during one week in three years in which no barrier contraceptives were used.

### Example 9

### Screening for DEFB126 Deletion Polymorphisms

Genotyping for the DEFB126 polymorphism finds use as one of a battery of standard diagnostice tests to perform in infertility programs. By establishing the DEFB 126 genotype of a patient early in the infertility evaluation, clinicians can obtain scientific evidence to justify rapid progression to directed interventions such as IUI and IVF, thus saving couples the time and expense of a protracted workup.

A clear example of the potential benefit is demonstrated by the case of unexplained infertility of donor D10 (Table 2, FIGS. 16 and 17). FIG. 16 provides data describing Human sperm were labeled with FITC-conjugated lectin ABA as described in FIG. 7. Differences in fluorescence can clearly be seen between sperm from the eight DEFB126 wild type donors (wt/wt and wt/del) and sperm from the six donors that carry only the DEFB126 gene variant (del/del). Sperm from donor D10 exhibit very low levels of bound lectin while sperm from donor D12 are brightly and uniformly labeled. FIG. 17 provides data describing HA penetration with sperm from D10 and D12. Penetration experiments were performed as described in FIGS. 14 and 15. Plot of sperm penetration values from del (pink) and wt (blue) males are reproduced from FIG. 15. HA penetration of sperm from D10 (light pink) and D12 (light blue) are shown in reference to average values for del and wt males respectively.

No fertility issues were identified in either D10 or his partner during a full infertility workup. Had the male been genotyped early in the fertility evaluation process the couple could have readily advanced to IUI, avoiding 18 months to 2 years of failed attempts.

Detection of the DEFB126 polymorphism also improves the prognostic power of routine measures of semen quality such as sperm motility, morphology, and count. Presently, these semen parameters while correlated with a man's fertilizing potential lack the specificity to be used reliably as predictors of male infertility (Guzick and Overstreet et al., (2001); Ombelet et al., (1997)). Our data suggests that the lack of a functional DEFB126 sperm coating protein is an important, but previously unexplained factor of subfertility that clouds what might be otherwise a clear interpretation of fertility status based on traditional clinical measures (Table 2). Measures of sperm density for donor D10 (considerably higher than the WHO cut-off value) and D12 (considerably lower than the WHO cut-off value) alone are poor predictors of fertility but following genotyping may help predict the fertilizing potential of men with a particular DEFB126 genotype.

### Example 10

### Detection of DEFB126 Glycoprotein in Semen

Lectins and/or antibodies can be used in conjunction with available technologies employed in home diagnostic kits for the detection of the DEFB126 glycoprotein in a semen sample. Our data shows that sperm from del/del donors have significantly less labeling with FITC-ABA than sperm from donors who posses the wildytpe gene (FIGS. 8 and 9). The difference in labeling intensity appears to be sufficiently large for determination of threshold values that can discriminate between DEFB 126 “pos” and DEFB 126 “neg” males. We have demonstrated previously in the macaque that lectin ABA as well as wheat germ agglutinin (WGA) strongly recognize DEFB126 on intact (and viable) sperm and on Western blots (Yudin et al., (2005)). Labeling with ABA requires that sperm initially be treated with sialidase, a step that can be performed with both living and fixed sperm (Yudin et al., (2005); Tollner et al., (2008)).

The lectins however will bind to the same classes of oligosaccharides that are potentially associated with glycolipids and other glyoproteins of the sperm glycocalyx. Antibodies may be warranted if greater specificity is required. Antibodies to sperm-specific proteins have been used to estimate sperm concentration using a lateral flow immunochromatographic home test device (“Sperm Check”; Klotz et al., (2008)). As general scheme for home detection of DEFB 126, a small portion of the semen sample would be added to saline containing a low % of detergent to release DEFB 126. As solubilized protein flows across a test pad, it hydrates a zone of gold-labeled anti-DEFB126 antibodies. DEFB126-antibody complexes would be captured and concentrated at a test-line by a secondary antibody adhered to the solid phase of the pad. We also imagine that a lectin-DEFB 126-antibody “sandwich” approach could also be adaptable to a test kit, where the lectins could be employed as a “sperm capture” strategy. ABA or WGA bound to solid supports would bind sperm via oligosaccharides on DEFB126. DEFB126 would be released from the sperm surface by treatment with phospholipase C or conditions of high salt and pH (Yudin et al., (2003); Tollner et al., (2009)). Mono- or polyclonal antibodies conjugated to biotin or enzymes would be applied to the solid phase for colorimetric detection of DEFB126. A similar sandwich assay greatly enhances the sensitivity and specificity of detection of serum mucins associated with pancreatic cancer by a monoclonal antibody (Neil Parker, (1998)).

### Example 11

### DEFB126 Can Be “Added Back” to the Sperm Surface, Restoring Sperm Function

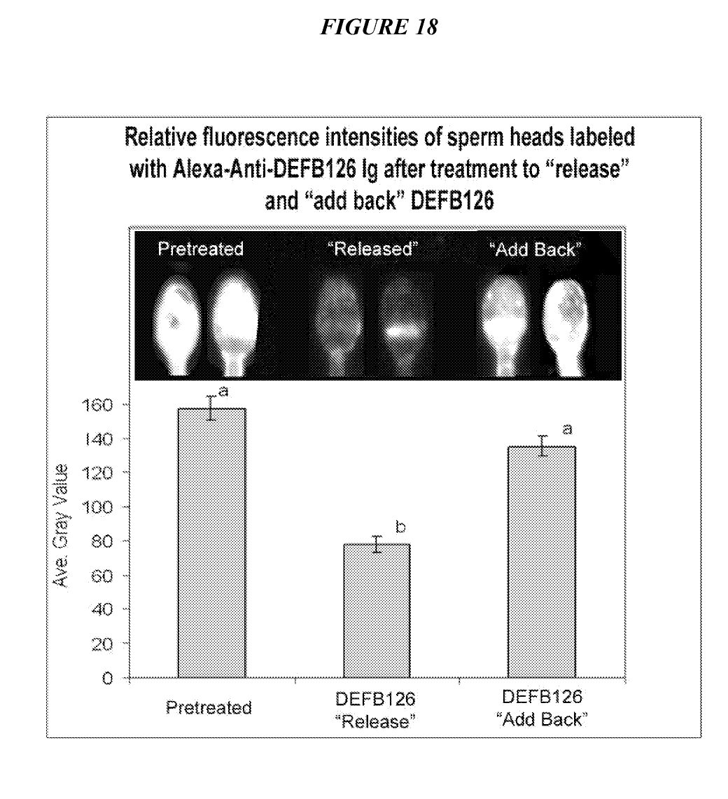

DEFB126 can be selectively released from sperm, purified, concentrated, and added back to the surface of sperm that have lost the surface coat following incubation in capacitating conditions (FIG. 18; Tollner et al., (2004)). Assessment of immunofluorescent labeling intensity of the heads of cynomolgus macaque sperm after treatment with 2 mM caffeine to release DEFB126 from sperm and caffeine-treated sperm following addition of add-back solution containing DEFB 126. Caffeine removes DEFB 126 from over the head and midpiece but does not induce capacitation. Immunofluorescent images of sperm heads labeled with anti-DEFB 126 Ig were at the same threshold level for all treatments and average pixel area and gray values determined for each sperm head using quantitative pixel analyses. Bar height represents the mean pixel intensity of sperm heads with treatment, and error bars represent SEM. Letters (a,b) above columns indicate significant differences between treatments (p<0.05) (FIG. modified from Tollner, (2004)).

The “add-back” of DEFB126 completely restores function of cynomolgus monkey sperm (Tollner et al., (2004; 2008a,b); FIG. 18), suggesting that its orientation upon re-insertion into the sperm membrane is the same as when originally adsorbed to the sperm surface.

It is herein demonstrated recently that purified DEFB 126 from cynomolgus macaques when added to human sperm from del/del males enhances the HA penetration ability of these DEFB126-deficient sperm. The penetration rate of “supplemented” sperm resembles that of sperm from wt males (FIG. 19A, B). Sperm from del/del donors were incubated with ˜5 uM cDEFB126 for 1 hr at 37C (orange). Control sperm received equal volume of saline solvent (pink). Sperm were washed by centrifugation, resuspended in BWW medium, and introduced into HA penetration chambers as described in FIGS. 14 and 15. Sperm from one del donor exhibited a doubling in the penetration rate while sperm from two other donors (one of which was D10) quadrupled in the rate of penetration. FIG. 19B shows the averaged plot of sperm from the two donors that exhibited the 4-fold response.

### Example 12

### Addition of Recombinant DEFB126 to Deficient Sperm

Our data demonstrates that the addition of DEFB 126 from cynomolgus macaques to the surface of human sperm from Del/Del donors elevates the level of function of these deficient sperm to levels observed for sperm from men who carry the wildtype gene (FIG. 18). This finding is both very exciting and highly unexpected. The implications of the data are clear—we can offer a treatment to men who have the Del/Del genotype or who are diagnosed with a deficit of the DEFB126 sperm coating protein. For the subfertile couple pursuing more cost effective clinical interventions, a glycosylated recombinant protein can be added to semen samples prior to vaginal artificial insemination to enhance the fertility of “del/del” sperm. Ultimately, the recombinant peptide can be concentrated in a vaginal foam or gel formulated to facilitate the adsorption of peptide to the sperm surface. The gel could be applied easily at home, enabling couples to achieve conception more naturally.

It is particularly surprising that the monkey homologue of DEFB 126 appears to work as effectively as the native human peptide. It is practically dogma in reproductive biology that even closely related species will differ considerably in their reproductive strategies as determined by one or some combination of differences in behavior, anatomy, and biochemical and molecular biology. This is particularly true of genes of proteins that mediate reproductive processes after copulation (i.e. gamete transport, storage, signal transduction and fertilization) which have been shown to be far more divergent then genes expressed in non-reproductive tissues (Swanson and Vacquier, 2002). DEFB126 is no exception to the expectation that reproductive proteins are rapidly evolving. Blast amino acid analysis comparing cynomolgus macaque and human DEFB126 shows that the full length proteins of these two primate species only share 71% sequence homology (FIG. 19A). Eliminating the signal sequence drops the homology to 66% (FIG. 19B). The homology is largely limited to the defensin core which is characterized by the conserved position of 6 cysteine residues and a few of the adjoining amino acids. The homology of the carboxyl end (aa 65-134: the glycoslyated end of DEFB126) drops to ˜50%. Our experiments demonstrate that the general properties of the molecule, a defensin with a glycosylated carboxyl extension, are sufficient to impart function. Our finding is surprising indeed, considering that oligosaccharide structures have also been shown to be primate species-specific and many sperm functions (such as oviductal binding, zona pelucida binding, sperm-egg fusion) in different mammalian species are mediated by completely different proteins.

A variety of different approaches can result in the production of an efficacious therapeutic DEFB126 construct. Glycopeptides with O-linked oligosaccharides are produced by the Sf9, Sf21 and TN-5B1-4 (High-Five) cell lines. For example, human interferon-α2 expressed in Sf9 cells was O-glycosylated at the same position as the natural interferon (Sugyiama et al., (1993)). The composition of the glycan can differ (GalNAc, and GalNAc+Gal not sialylated) but replicating the exact oligosaccharide sequence and branching may not be necessary so long as these glycans are adequately negatively charged. Similarly, human interleukin-2 expressed in Sf21 cell contained an O-glycan of Gal-3Gal NAc (Grabenhorst et al., (1993)). In contrast, high-five cells can produce recombinant peptides with terminal galactose and α2,6 and α2,3 sialic acids (Davis et al., (1993); Davis and Wood, (1995)). Insect cell recombinant products can provide us with a range of O-glycan variants, with some that will closely resemble native DEFB 126 with respect to the degree of glycosylation and negative charge groups. In addition, recombinant expression in yeast and mammalian cell lines will provide alternative approaches for engineering a glycosylated peptide. In principle, it should be possible to improve upon nature's “design” by engineering a molecule that retains the defensin motif critical for binding to the sperm surface but with a more thoroughly glycosylated and more negatively charged carboxyl tail.

It is understood that the examples and embodiments described herein are for illustrative purposes only and that various modifications or changes in light thereof will be suggested to persons skilled in the art and are to be included within the spirit and purview of this application and scope of the appended claims. All publications, patents, patent applications and accession numbers cited herein are hereby incorporated by reference in their entirety for all purposes.

### REFERENCES

Aitken R J, Bowie H, Buckingham D, Harkiss D, Richardson D W, West K M (1992) Sperm penetration into a hyaluronic acid polymer as a means of monitoring functional competence. J Androl 13: 44-54.
Aitken R J (2006) Sperm function tests and fertility. Int J Androl 29: 69-75; discussion 105-108.
Arienti G, Polci A, Carlini E and Palmerini C A (1997) Transfer of CD26/dipeptidyl peptidase IV (E.C. 3.5.4.4) from prostasomes to sperm. FEBS Lett, 410: 343-6.
Cooper T G (1998). Interactions between epididymal secretions and spermatozoa. J Reprod Fertil Suppl, 53: 119-136.
Chen G N, Yudin A I, Li M W, Vines C A, Overstreet J W (1999). Hyaluronic acid and the cumulus extracellular matrix induce increases in intracellular calcium in macaque sperm via the plasma membrane protein PH-20. Zygote 7: 211-222
Cuasnicu P S, Ellerman D A, Cohen D J, Busso D, Morgenfeld M M and Da Ros V G (2001). Molecular mechanisms involved in mammalian gamete fusion. Arch Med Res, 32: 614-618.
Davis T R, Wickham T J, McKenna K A, Granados R R, Shuler M L and Wood H A (1993). Comparative recombinant protein production of eight insect cell lines. In Vitro Cell Dev Biol Anim, 29A: 388-390.
Davis T R, Wood H A (1995). Intrinsic glycosylation potentials of insect cell cultures and insect larvae. In Vitro Cell Dev Biol Anim, 31: 659-663.
de Lamirande E, Yoshida K, Yoshiike T M, Iwamoto T, Gagnon C (2001). Semenogelin, the main protein of semen coagulum, inhibits human sperm capacitation by interfering with the superoxide anion generated during this process. J Androl, 22: 672-679.
Frenette G, Sullivan R (2001). Prostasome-like particles are involved in the transfer of P25b from the bovine epididymal fluid to the sperm surface. Mol Reprod Dev, 59: 115-121.
Gatej I, Popa M, Rinaudo M (2005) Role of the pH on Hyaluronan Behavior in Aqueous Solution. Biomacromolecules 6: 61-67.
Gatti J L, Druart X, Syntin P, Guerin Y, Dacheux J L and Dacheux F (2000). Biochemical characterization of two ram cauda epididymal maturation-dependent sperm glycoproteins. Biol Reprod, 62: 950-958.
Gatti J L, Castella S, Dacheux F, Ecroyd H, Metayer S, Thimon V and Dacheux J L (2004). Post-testicular sperm environment and fertility. Anim Reprod Sci, 82-83: 321-339.
Gipson I K, Ho S B, Spurr-Michaud S J, Tisdale A S, Zhan Q, Torlakovic E, Pudney J, Anderson D J, Toribara N W, Hill J A, 3rd (1997) Mucin genes expressed by human female reproductive tract epithelia. Biol Reprod 56: 999-1011.
Grabenhorst E, Hofer B, Nimtz M, Jager V and Conradt H S (1993). Biosynthesis and secretion of human interleukin 2 glycoprotein variants from baculovirus-infected Sf21 cells. Characterization of polypeptides and posttranslational modifications. Eur J Biochem, 215: 189-97.
Guzick D S, Overstreet J W, Factor-Litvak P, Brazil C K, Nakajima S T, Coutifaris C, Carson S A, Cisneros P, Steinkampf M P, Hill J A, Xu D, Vogel D L (2001) Sperm morphology, motility, and concentration in fertile and infertile men. N Engl J Med 345: 1388-1393.
Holland M K and Orgebin-Crist M C (1988). Characterization and hormonal regulation of protein synthesis by the murine epididymis. Biol Reprod, 38: 487-496.
Holt W V 1980. Surface-bound sialic acid on ram and bull spermatozoa: deposition during epididymal transit and stability during washing. Biol Reprod, 23: 847-857.
Julenius K, Molgaard A, Gupta R and Brunak S (2005). Prediction, conservation analysis, and structural characterization of mammalian mucin-type O-glycosylation sites. Glycobiology, 15: 153-64.
Kirchhoff C, Pera I, Derr P, Yeung C H, Cooper T (1997). The molecular biology of the sperm surface. Post-testicular membrane remodelling. Adv Exp Med Biol, 424: 221-232.
Klotz K L, Coppola M A, Labrecque M, Brugh V M 3rd, Ramsey K, Kim K A, Conaway M R, Howards S S, Flickinger C J, Herr J C. (2008) Clinical and consumer trial performance of a sensitive immunodiagnostic home test that qualitatively detects low concentrations of sperm following vasectomy. J Urol. 180:2569-2576.
Kohane A C, Cameo M S, Pineiro L, Garberi J C, Blaquier J A (1980). Distribution and site of production of specific proteins in the rat epididymis. Biol Reprod, 23: 181-187.
Lagow E, DeSouza M M, Carson D D (1999) Mammalian reproductive tract mucins. Hum Reprod Update 5: 280-292.
Neuwinger J, Cooper T G, Knuth U A, Nieschlag E (1991) Hyaluronic acid as a medium for human sperm migration tests. Hum Reprod 6: 396-400.
Ombelet W, Bosmans E, Janssen M, Cox A, Vlasselaer J, Gyselaers W, Vandeput H, Gielen J, Pollet H, Maes M, Steeno O, Kruger T. (1997) Semen parameters in a fertile versus subfertile population: a need for change in the interpretation of semen testing. Hum Reprod 12:987-993.
Parker N (1998) Lectin/Antibody “Sandwich” ELISA for Quantification of Circulating Mucin as a Diagnostic Test for Pancreatic Cancer. From: Methods in Molecular Medicine, Vol 9 Lectin Methods and Protocols. Eds. J M Rhodes and J D Milton. Humana Press Inc, Totowa, N.J.
Patil A A, Cai Y, Sang Y, Blecha F, Zhang G (2005). Cross-species analysis of the mammalian beta-defensin gene family: presence of syntenic gene clusters and preferential expression in the male reproductive tract. Physiol Genomics, 23: 5-17.
Perry A C, Jones R, Moisyadi S, Coadwell J, Hall L (1999) The novel epididymal secretory protein ESP13.2 in Macaca fascicularis. Biol Reprod 61: 965-972.
Radhakrishnan Y, Hamil K G, Yenugu S, Young S L, French F S, Hall S H (2005) Identification, characterization, and evolution of a primate beta-defensin gene cluster. Genes Immun 6: 203-210.
Regalado F, Nieto A (1989). In vitro biosynthesis and secretion of rabbit epididymal secretory proteins: regulation by androgens. J Exp Zool, 250: 214-218.
Swanson W J, Vacquier V D (2002) The rapid evolution of reproductive proteins. Nat Reviews. 3:137-144.
Sugyiama K, Ahorn H, Maurer-Fogy I, Voss T (1993). Expression of human interferon-alpha 2 in Sf9 cells. Characterization of O-linked glycosylation and protein heterogeneities. Eur J Biochem, 217: 921-7.
Tang S, Garrett C, Baker H W (1999) Comparison of human cervical mucus and artificial sperm penetration media. Hum Reprod 14: 2812-2817.
Tezon J G, Ramella E, Cameo M S, Vazquez M H, Blaquier J A (1985). Immunochemical localization of secretory antigens in the human epididymis and their association with spermatozoa. Biol Reprod, 32: 591-597.
Tollner T L, Yudin A I, Treece C A, Overstreet J W, Cherr G N (2004) Macaque sperm release ESP13.2 and PSP94 during capacitation: The absence of ESP13.2 is linked to sperm-zona recognition and binding. Mol Reprod Dev 69: 325-337.
Tollner T L, Yudin A I, Tarantal A F, Treece C A, Overstreet J W, Cherr G N (2008a) Beta-defensin 126 on the surface of macaque sperm mediates attachment of sperm to oviductal epithelia. Biol Reprod 78: 400-412.
Tollner T L, Yudin A I, Treece C A, Overstreet J W, Cherr G N (2008b) Macaque sperm coating protein DEFB126 facilitates sperm penetration of cervical mucus. Hum Reprod. 23:2523-34.
Tollner T, VandeVoort C, Yudin A, Treece C, Overstreet J, Cherr G (2009) Release of DEFB126 From Macaque Sperm and Completion of Capacitation Are Triggered by Conditions that Simulate Periovulatory Oviductal Fluid. Molec. Reprod. Dev 76:431-43.
Yanagimachi R (1994). Mammalian fertilization. In: The Physiology of Reproduction (Eds E. Knobil and J. D. Neill),. Raven Press, New York. pp. 189-317.
Yudin A I, Tollner T L, Li M W, Treece C A, Overstreet J W, Cherr G N (2003) ESP13.2, a member of the beta-defensin family, is a macaque sperm surface-coating protein involved in the capacitation process. Biol Reprod 69: 1118-1128.
Yudin A I, Treece C A, Tollner T L, Overstreet J W, Cherr G N (2005a) The carbohydrate structure of DEFB126, the major component of the cynomolgus Macaque sperm plasma membrane glycocalyx. J Membr Biol 207: 119-129.
Yudin A I, Generao S E, Tollner T L, Treece C A, Overstreet J W, Cherr G N (2005b) Beta-defensin 126 on the cell surface protects sperm from immunorecognition and binding of anti-sperm antibodies. Biol Reprod 73: 1243-1252.
Yudin A, Tollner T, Treece C, Kays R, Cherr G, Overstreet J, Bevins C (2008) Beta-defensin 22 is a major component of the mouse sperm glyocalyx. Reproduction 136:753-65.
Zanich A, Pascall J C and Jones R (2003). Secreted epididymal glycoprotein 2D6 that binds to the sperm's plasma membrane is a member of the beta-defensin superfamily of pore-forming glycopeptides. Biol Reprod, 69: 1831-42.
Zhou C X, Zhang Y L, Xiao L, Zheng M, Leung K M, Chan M Y, Lo P S, Tsang L L, Wong H Y, Ho L S, Chung Y W and Chan H C (2004). An epididymis-specific beta-defensin is important for the initiation of sperm maturation. Nat Cell Biol, 6: 458-64.

### Informal Sequence Listing

SEQ ID NO:1-wild-type human DEFB-126 nucleotide sequence spanning region encompassing DEFB-126 deletion polymorphism
tcctacccccgtttc
SEQ ID NO:2-wild-type human DEFB-126 nucleotide sequence spanning region encompassing DEFB-126 deletion polymorphism
atggctcctacccccgtttctccca
SEQ ID NO:3-wild-type human DEFB-126 polypeptide C-terminus sequence
PVSPTG

SEQ ID NO:4-wild-type DEFB-126 nucleic acid sequence Gen Bank Accession No. NM_{—}030931

1 agaacccact gcctcctgat gaagtcccta ctgttcaccc ttgcagtttt tatgctcctg

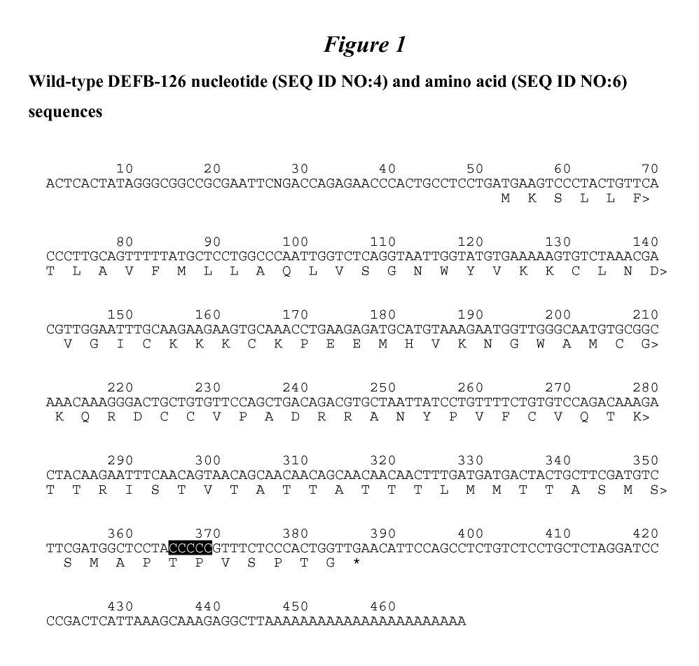

61 gcccaattgg tctcaggtaa ttggtatgtg aaaaagtgtc taaacgacgt tggaatttgc 121 aagaagaagt gcaaacctga agagatgcat gtaaagaatg gttgggcaat gtgcggcaaa 181 caaagggact gctgtgttcc agctgacaga cgtgctaatt atcctgtttt ctgtgtccag 241 acaaagacta caagaatttc aacagtaaca gcaacaacag caacaacaac tttgatgatg 301 actactgctt cgatgtcttc gatggctcct acccccgttt ctcccactgg ttgaacattc 361 cagcctctgt ctcctgctct aggatccccg actcattaaa gcaaagaggc tta
SEQ ID NO:5-wild-type DEFB-126 nucleic acid sequence as depicted in FIG. 1.

ACTCACTATAGGGCGGCCGCGAATTCNGACCAGAGAACCCACTGCCTCCTGATGAAGTCCCTACTGTTCA CCCTTGCAGTTTTTATGCTCCTGGCCCAATTGGTCTCAGGTAATTGGTATGTGAAAAAGTGTCTAAACGA CGTTGGAATTTGCAAGAAGAAGTGCAAACCTGAAGAGATGCATGTAAAGAATGGTTGGGCAATGTGCGGC AAACAAAGGGACTGCTGTGTTCCAGCTGACAGACGTGCTAATTATCCTGTTTTCTGTGTCCAGACAAAGA CTACAAGAATTTCAACAGTAACAGCAACAACAGCAACAACAACTTTGATGATGACTACTGCTTCGATGTC TTCGATGGCTCCTACCCCCGTTTCTCCCACTGGTTGAACATTCCAGCCTCTGTCTCCTGCTCTAGGATCC CCGACTCATTAAAGCAAAGAGGCTT
SEQ ID NO:6 -wild-type DEFB-126 amino acid sequence from Homo sapiens
Gen Bank Accession No. NP_{—}112193

1 MKSLLFTLAV FMLLAQLVSG NWYVKKCLND VGICKKKCKP EEMHVKNGWA MCGKQRDCCV 61 PADRRANYPV FCVQTKTTRI STVTATTATT TLMMTTASMS SMAPTPVSPT G
SEQ ID NO:7 -wild-type DEFB-126 amino acid sequence from Hylobates lar
Gen Bank Accession No. A4H245.1
MKSLLFTLAVFMLLAQLVSGNWYVKKCLNDVG ICKKKCKPEELHVKNGRAMCGKQRDCCVPADK RANYPAFCVQTKTTRTSTVTATAAT - - - - - - - - TTTLVMTTASMSSMA - - - - - - - - - - - - - PT PVSPTS -
SEQ ID NO:8-wild-type DEFB-126 amino acid sequence from Gorilla gorilla
Gen Bank Accession No. A4H243.1
MKSLLFTLAVFMLLAQLVSGNWYVKKCLNDVG ICKKKCKPEEMHVKNGWAMCGKQRDCCVPADR RANYPAFCVQTKTTRTSTVTATTAT - - - - - - - - - TTLMMTTASMSLMA - - - - - - - - - - - - - - PT PVSPTG -
SEQ ID NO:9-wild-type DEFB-126 amino acid sequence from Pan troglodytes
Gen Bank Accession No. XP _{—}514453
MKSLLFTLAVFMLLAQLVSGNWYVKKCLNDVG ICKKKCKPGEMHIKNGWATCGKQRDCCVPADR RANYPAFCVQTKTTRTSTVTAR - - - - - - - - - - - - TTLMVTTASMSSMA - - - - - - - - - - - - - - PT PVSPTG -
SEQ ID NO:10 -wild-type DEFB-126 amino acid sequence from Macaca fascicularis
Gen Bank Accession No. CAL68961.1
MKSLLFTLAVFMLLAQLVSGNLYVKRCLNDIG ICKKTCKPEEVRSEHGWVMCGKRKACCVPADK RSAYPSFCVHSKTTKTSTVTARATATTATTAT AATPLMISNGLISLMSYDGRYPCFSHYLNIPA SVSCSRS
SEQ ID NO:11 -wild-type DEFB-126 amino acid sequence from Pongo pygmaeus
Gen Bank Accession No. A4H244.1
MKSLLFTLAVFMLLAQLVSGSWYVKKCLNDVG ICKKKCKPEELHVKNGWAMCGKQRDCCVPADK RANYPAFCVQTKTTRTSTVTATTATRATTAT - - TTTLMMTTASMSSMT - - - - - - - - - - - - - - PT PVSPTG -
SEQ ID NO:12-wild-type DEFB-126 consensus sequence comparing Human, Hylobates lar, Gorilla gorilla, Pan troglodytes, Macaca fascicularis, and Pongo pygmaeus.
MKSLLFTLAVFMLLAQLVSGNWYVKKCLNDVG ICKKKCKPEE - HVKNGWAMCGKQRDCCVPAD. RANYPAFCVQTKTTRTSTVTATTAT - - - T - - T ATTTLMMTTASMSSMAYDGRYPCFSHYLNIPT PVSPTGS
SEQ ID NO:13 -DEFB-126 deletion polymorphism nucleic acid sequence
Gen Bank Accession No. AK225987

1 atagagactt ctggactcta tagaacccac tgcctcctga tgaagtccct actgttcacc 61 cttgcagttt ttatgctcct ggcccaattg gtctcaggta attggtatgt gaaaaagtgt 121 ctaaacgacg ttggaatttg caagaagaag tgcaaacctg aagagatgca tgtaaagaat 181 ggttgggcaa tgtgcggcaa acaaagggac tgctgtgttc cagctgacag acgtgctaat 241 tatcctgttt tctgtgtcca gacaaagact acaagaattt caacagtaac agcaacaaca 301 gcaacaacaa ctttgatgat gactactgct tcgatgtctt cgatggctcc tacccgtttc 361 tcccactggt tgaacattcc agcctctgtc tcctgctcta ggatccccga ctcattaaag 421 caaagaggct taaaaaaaaa aaaaaaaaaa aaaaaaaaaa a
SEQ ID NO:14-DEFB-126 deletion polymorphism nucleic acid sequence as depicted in FIG. 2.
ACTCACTATAGGGCGGCCGCGAATTCNGACCAGAGAACCCACTGCCTCCTGATGAAGTCCCTAC TGTTCACCCTTGCAGTTTTTATGCTCCTGGCCCAATTGGTCTCAGGTAATTGGTATGTGAAAAA GTGTCTAAACGACGTTGGAATTTGCAAGAAGAAGTGCAAACCTGAAGAGATGCATGTAAAGAAT GGTTGGGCAATGTGCGGCAAACAAAGGGACTGCTGTGTTCCAGCTGACAGACGTGCTAATTATC CTGTTTTCTGTGTCCAGACAAAGACTACAAGAATTTCAACAGTAACAGCAACAACAGCAACAAC AACTTTGATGATGACTACTGCTTCGATGTCTTCGATGGCTCCTACCCGTTTCTCCCACTGGTTG AACATTCCAGCCTCTGTCTCCTGCTCTAGGATCCCCGACTCATTAAAGCAAAGAGGCTTAAAAA
SEQ ID NO:15 -DEFB-126 deletion polymorphism nucleic acid sequence (reverse compliment)
Gen Bank Accession No. CO408416

1 agacagaggc tggaatgtca accagtggga gaaacgggta ggagccatcg aagacatcga 61 agcagtagtc atcatcaaag ttgttgttgc tgttgttgct gttactgttg aaattcttgt 121 agtctttgtc tggacacaga aaacaggata attagcacgt ctgtcagctg gaacacagca 181 gtccctttgt ttgccgcaca ttgcccaacc attctttaca tgcatctctt caggtttgca 241 cttcttcttg caaattccaa cgtcgtttag acactttttc acataccaat tacctgagac 301 caattgggcc aggagcataa aaactgcaag ggtgaacagt agggacttca tcaggaggca 361 gtgggttcta tagagtccag aagtctctat tcagtatgac tctgaacaca gatctttatt 421 gtccttcccc c
SEQ ID NO:16-variant DEFB-126 deletion polymorphism amino acid sequence. n=1 to 50 amino acids.
MKSLLFTLAV FMLLAQLVSG NWYVKKCLND VGICKKKCKP EEMHVKNGWA MCGKQRDCCV PADRRANYPV FCVQTKTTRI STVTATTATT TLMMTTASMS SMAPTRFSHWLNIPASVSCSRIPDSLKQRGL(K)ₙ
SEQ ID NO:17-variant DEFB-126 deletion polymorphism C-terminal amino acid sequence. n=1 to 50 amino acids.
VPADRRANYPVFCVQTKTTRISTVTATTATTTLMMTTASMSSMAPTRFSHWLNIPASVSCSRIPDSLKQRGL(K)ₙ
SEQ ID NO:18-variant DEFB-126 deletion polymorphism C-terminal amino acid sequence. N=1 to 50 amino acids.
RFSHWLNIPASVSCSRIPDSLKQRGL (K)ₙ

1. A method for determining whether an individual has an increased risk of infertility comprising: determining the DEFB-126 alleles of the individual within the subsequence TCCTACCCCCGTTTC (SEQ ID NO:1) of a nucleic acid encoding DEFB-126, wherein the presence of five contiguous cytosines “CCCCC” at positions 6-10 within the subsequence is indicative of normal fertility and the presence of at most three contiguous cytosines “CCC” within positions 6-10 of the subsequence is indicative of an increased probability of infertility.

2. The method of claim 1, wherein the subsequence is ATGGCTCCTACCCCCGTTTCTCCA (SEQ ID NO:2) and the presence of five contiguous cytosines “CCCCC” at positions 11-15 within the subsequence is indicative of normal fertility and the presence of at most three contiguous cytosines “CCC” within positions 11-15 of the subsequence is indicative of an increased probability of infertility.

3. The method of claim 1, wherein the individual is human.

4. The method of claim 1, wherein the individual is male.

5. The method of claim 1, wherein the nucleic acid is DNA.

6. (canceled)

7. The method of claim 1, wherein the nucleic acid encoding DEFB-126 shares at least 95% sequence identity to SEQ ID NO:4.

8. The method of claim 1, wherein the DEFB-126 alleles are detected by an amplification reaction using one or more polynucleotides that distinguish between alleles within the subsequence TCCTACCCCCGTTTC (SEQ ID NO:1) of a nucleic acid encoding DEFB-126.

9. The method of claim 8, wherein the amplification reaction is selected from the group consisting of polymerase chain reaction (PCR), strand displacement amplification (SDA), nucleic acid sequence based amplification (NASBA), rolling circle amplification (RCA), T7 polymerase mediated amplification, T3 polymerase mediated amplification, and SP6 polymerase mediated amplification.

10. The method of claim 1, wherein the DEFB-126 alleles are detected by hybridization using one or more polynucleotides that distinguish between alleles within the subsequence TCCTACCCCCGTTTC (SEQ ID NO:1) of a nucleic acid encoding DEFB-126.

11. The method of claim 1, wherein the DEFB-126 alleles are detected by sequencing a subsequence of DEFB-126, the subsequence comprising the nucleic acid sequence TCCTACCCCCGTTTC (SEQ ID NO:1).

12. The method of claim 1, wherein the DEFB-126 alleles are detected by restriction fragment length polymorphism.

13. (canceled)

14. A method for determining whether an individual has an increased probability of infertility comprising obtaining a biological sample from the individual and determining the presence of a DEFB 126 polypeptide in the sample, wherein the presence of a DEFB-126 polypeptide is indicative of normal fertility and the absence of a DEFB-126 polypeptide is indicative of an increased probability of infertility.

15-21. (canceled)

22. A method for determining whether an individual has an increased probability of infertility comprising obtaining a sperm sample from the individual and contacting the sample with a lectin that selectively binds Galactose-GalNAc or sialic acid, wherein a reduced binding level of the lectin in comparison to a normal control is indicative of an increased probability of infertility.

23-24. (canceled)

25. A kit for determining whether an individual has an increased probability of infertility, the kit comprising at least one polynucleotide that distinguishes the DEFB-126 alleles of the individual within the subsequence TCCTACCCCCGTTTC (SEQ ID NO:1), and instructions indicating that the presence of five contiguous cytosines “CCCCC” at positions 6 10 within the subsequence is indicative of normal fertility and the presence of at most three contiguous cytosines “CCC” within positions 6-10 of the subsequence is indicative of an increased probability of infertility.

26. A kit for determining whether an individual has an increased probability of infertility, the kit comprising at least one antibody that recognizes a DEFB-126 polypeptide, and instructions indicating that the presence of a DEFB-126 polypeptide is indicative of normal fertility and the absence of a DEFB-126 polypeptide is indicative of an increased probability of infertility.

27. (canceled)

28. A kit for determining whether an individual has an increased probability of infertility, the kit comprising at least one lectin that specifically binds to DEFB-126 and instructions indicating that the presence of a DEFB-126 polypeptide is indicative of normal fertility and the absence of a DEFB-126 polypeptide is indicative of an increased probability of infertility.

29-31. (canceled)

32. A kit for determining whether an individual has an increased probability of infertility, the kit comprising poly-L-lysine and instructions indicating that the presence of a DEFB-126 polypeptide is indicative of normal fertility and the absence of a DEFB-126 polypeptide is indicative of an increased probability of infertility.

33. (canceled)

34. A method for restoring sperm functionality from an individual who expresses insufficient levels of functional DEFB-126 to effect conception, comprising contacting a sperm sample obtained from said individual with a functional DEFB-126 polypeptide.

35-45. (canceled)

46. A composition comprising a functional DEFB-126 polypeptide and a pharmaceutically acceptable carrier.

47-55. (canceled)


SEQUENCE LISTING (50 sequences)

SEQ ID NO: 1 - DNA, 15 bp, Artificial Sequence
  synthetic wild-type human DEFB-126 nucleotide sequence spanning region encompassing DEFB-126 deletion polymorphism, DEFB-126 subsequence
tcctacccccgtttc

SEQ ID NO: 2 - DNA, 25 bp, Artificial Sequence
  synthetic wild-type human DEFB-126 nucleotide, DEFB-126 subsequence sequence spanning region encompassing DEFB-126 deletion polymorphism
atggctcctacccccgtttctccca

SEQ ID NO: 3 - protein, 6 aa, Artificial Sequence
  synthetic wild-type human DEFB-126 polypeptide C-terminal sequence
PVSPTG

SEQ ID NO: 4 - DNA, 413 bp, Homo sapiens
  human wild-type beta defensin 126 (DEFB-126, DEFB126, DEFB-26, DEFB26, hBD-26) preprotein, epididymal secretory protein ESP13.2, bA530N10.1, C20orf8 cDNA
agaacccactgcctcctgatgaagtccctactgttcacccttgcagtttttatgctcctggcccaattggtctcaggtaattggtatgtgaaaaagtgtctaaacgacgttggaatttgcaagaagaagtgcaaacctgaagagatgcatgtaaagaatggttgggcaatgtgcggcaaacaaagggactgctgtgttccagctgacagacgtgctaattatcctgttttctgtgtccagacaaagactacaagaatttcaacagtaacagcaacaacagcaacaacaactttgatgatgactactgcttcgatgtcttcgatggctcctacccccgtttctcccactggttgaacattccagcctctgtctcctgctctaggatccccgactcattaaagcaaagaggctta

SEQ ID NO: 5 - DNA, 468 bp, Homo sapiens
  human wild-type beta defensin 126 (DEFB-126)
actcactatagggcggccgcgaattcngaccagagaacccactgcctcctgatgaagtccctactgttcacccttgcagtttttatgctcctggcccaattggtctcaggtaattggtatgtgaaaaagtgtctaaacgacgttggaatttgcaagaagaagtgcaaacctgaagagatgcatgtaaagaatggttgggcaatgtgcggcaaacaaagggactgctgtgttccagctgacagacgtgctaattatcctgttttctgtgtccagacaaagactacaagaatttcaacagtaacagcaacaacagcaacaacaactttgatgatgactactgcttcgatgtcttcgatggctcctacccccgtttctcccactggttgaacattccagcctctgtctcctgctctaggatccccgactcattaaagcaaagaggcttaaaaaaaaaaaaaaaaaaaaaaa

SEQ ID NO: 6 - protein, 111 aa, Homo sapiens
  human wild-type beta defensin 126 (DEFB-126, DEFB126, DEFB-26, DEFB26, hBD-26) preprotein, epididymal secretory protein ESP13.2, bA530N10.1, C20orf8
MKSLLFTLAVFMLLAQLVSGNWYVKKCLNDVGICKKKCKPEEMHVKNGWAMCGKQRDCCVPADRRANYPVFCVQTKTTRISTVTATTATTTLMMTTASMSSMAPTPVSPTG

SEQ ID NO: 7 - protein, 112 aa, Hylobates lar
  common gibbon wild-type beta defensin 126 (DEFB-126, DEFB126, DEFB-26, DEFB26)
MKSLLFTLAVFMLLAQLVSGNWYVKKCLNDVGICKKKCKPEELHVKNGRAMCGKQRDCCVPADKRANYPAFCVQTKTTRTSTVTATAATTTTLVMTTASMSSMAPTPVSPTS

SEQ ID NO: 8 - protein, 111 aa, Gorilla gorilla
  Western lowland gorilla wild-type beta defensin 126 (DEFB-126, DEFB126, DEFB-26, DEFB26)
MKSLLFTLAVFMLLAQLVSGNWYVKKCLNDVGICKKKCKPEEMHVKNGWAMCGKQRDCCVPADRRANYPAFCVQTKTTRTSTVTATTATTTLMMTTASMSLMAPTPVSPTG

SEQ ID NO: 9 - protein, 108 aa, Pan troglodytes
  chimpanzee wild-type beta defensin 126 (DEFB-126, DEFB126, DEFB-26, DEFB26)
MKSLLFTLAVFMLLAQLVSGNWYVKKCLNDVGICKKKCKPGEMHIKNGWATCGKQRDCCVPADRRANYPAFCVQTKTTRTSTVTARTTLMVTTASMSSMAPTPVSPTG

SEQ ID NO: 10 - protein, 135 aa, Macaca fascicularis
  crab-eating macaque wild-type beta defensin 126 (DEFB-126, DEFB126, DEFB-26, DEFB26)
MKSLLFTLAVFMLLAQLVSGNLYVKRCLNDIGICKKTCKPEEVRSEHGWVMCGKRKACCVPADKRSAYPSFCVHSKTTKTSTVTARATATTATTATAATPLMISNGLISLMSYDGRYPCFSHYLNIPASVSCSRS

SEQ ID NO: 11 - protein, 118 aa, Pongo pygmaeus
  Bornean orangutan wild-type beta defensin 126 (DEFB-126, DEFB126, DEFB-26, DEFB26)
MKSLLFTLAVFMLLAQLVSGSWYVKKCLNDVGICKKKCKPEELHVKNGWAMCGKQRDCCVPADKRANYPAFCVQTKTTRTSTVTATTATRATTATTTTLMMTTASMSSMTPTPVSPTG

SEQ ID NO: 12 - protein, 129 aa, Artificial Sequence
  synthetic wild-type DEFB-126 protein primate consensus sequence
MKSLLFTLAVFMLLAQLVSGNWYVKKCLNDVGICKKKCKPEEHVKNGWAMCGKQRDCCVPADXRANYPAFCVQTKTTRTSTVTATTATTTATTTLMMTTASMSSMAYDGRYPCFSHYLNIPTPVSPTGS

SEQ ID NO: 13 - DNA, 461 bp, Homo sapiens
  human DEFB-126 with deletion polymorphism frame shift
atagagacttctggactctatagaacccactgcctcctgatgaagtccctactgttcacccttgcagtttttatgctcctggcccaattggtctcaggtaattggtatgtgaaaaagtgtctaaacgacgttggaatttgcaagaagaagtgcaaacctgaagagatgcatgtaaagaatggttgggcaatgtgcggcaaacaaagggactgctgtgttccagctgacagacgtgctaattatcctgttttctgtgtccagacaaagactacaagaatttcaacagtaacagcaacaacagcaacaacaactttgatgatgactactgcttcgatgtcttcgatggctcctacccgtttctcccactggttgaacattccagcctctgtctcctgctctaggatccccgactcattaaagcaaagaggcttaaaaaaaaaaaaaaaaaaaaaaaaaaaaaa

SEQ ID NO: 14 - DNA, 466 bp, Homo sapiens
  human DEFB-126 with deletion polymorphism frame shift
actcactatagggcggccgcgaattcngaccagagaacccactgcctcctgatgaagtccctactgttcacccttgcagtttttatgctcctggcccaattggtctcaggtaattggtatgtgaaaaagtgtctaaacgacgttggaatttgcaagaagaagtgcaaacctgaagagatgcatgtaaagaatggttgggcaatgtgcggcaaacaaagggactgctgtgttccagctgacagacgtgctaattatcctgttttctgtgtccagacaaagactacaagaatttcaacagtaacagcaacaacagcaacaacaactttgatgatgactactgcttcgatgtcttcgatggctcctacccgtttctcccactggttgaacattccagcctctgtctcctgctctaggatccccgactcattaaagcaaagaggcttaaaaaaaaaaaaaaaaaaaaaaa

SEQ ID NO: 15 - DNA, 431 bp, Homo sapiens
  human DEFB-126 with deletion polymorphism frame shift (reverse complement), EST00015
agacagaggctggaatgtcaaccagtgggagaaacgggtaggagccatcgaagacatcgaagcagtagtcatcatcaaagttgttgttgctgttgttgctgttactgttgaaattcttgtagtctttgtctggacacagaaaacaggataattagcacgtctgtcagctggaacacagcagtccctttgtttgccgcacattgcccaaccattctttacatgcatctcttcaggtttgcacttcttcttgcaaattccaacgtcgtttagacactttttcacataccaattacctgagaccaattgggccaggagcataaaaactgcaagggtgaacagtagggacttcatcaggaggcagtgggttctatagagtccagaagtctctattcagtatgactctgaacacagatctttattgtccttccccc

SEQ ID NO: 16 - protein, 181 aa, Homo sapiens
  human DEFB-126 with deletion polymorphism frame shift, DEFB-126 variant polypeptide with extended C-terminal region
MKSLLFTLAVFMLLAQLVSGNWYVKKCLNDVGICKKKCKPEEMHVKNGWAMCGKQRDCCVPADRRANYPVFCVQTKTTRISTVTATTATTTLMMTTASMSSMAPTRFSHWLNIPASVSCSRIPDSLKQRGLKKKKKKKKKKKKKKKKKKKKKKKKKKKKKKKKKKKKKKKKKKKKKKKKKK

SEQ ID NO: 17 - protein, 122 aa, Artificial Sequence
  synthetic human DEFB-126 with deletion polymorphism frame shift C-terminal sequence, DEFB-126 variant polypeptide C-terminal sequence
VPADRRANYPVFCVQTKTTRISTVTATTATTTLMMTTASMSSMAPTRFSHWLNIPASVSCSRIPDSLKQRGLKKKKKKKKKKKKKKKKKKKKKKKKKKKKKKKKKKKKKKKKKKKKKKKKKK

SEQ ID NO: 18 - protein, 76 aa, Artificial Sequence
  synthetic human DEFB-126 with deletion polymorphism frame shift C-terminal sequence, DEFB-126 variant polypeptide C-terminal sequence
RFSHWLNIPASVSCSRIPDSLKQRGLKKKKKKKKKKKKKKKKKKKKKKKKKKKKKKKKKKKKKKKKKKKKKKKKKK

SEQ ID NO: 19 - protein, 34 aa, Artificial Sequence
  synthetic beta-defensin six-cysteine motif, canonical core of cysteine residues
CXXXXXXCXXXXCXXXXXXXXXXXXCXXXXXXCC

SEQ ID NO: 20 - protein, 6 aa, Artificial Sequence
  synthetic human DEFB-126 with deletion polymorphism frame shift extended C-terminal sequence beginning motif, DEFB-126 variant polypeptide extended C-terminal sequence beginning motif
SMSXMX

SEQ ID NO: 21 - protein, 138 aa, Homo sapiens
  human DEFB-126 with deletion polymorphism frame shift, DEFB-126 variant polypeptide with extended C-terminal region
MKSLLFTLAVFMLLAQLVSGNWYVKKCLNDVGICKKKCKPEEMHVKNGWAMCGKQRDCCVPADRRANYPVFCVQTKTTRISTVTATTATTTLMMTTASMSSMAPTRFSHWLNIPASVSCSRIPDSLKQRGLKKKKKKK

SEQ ID NO: 22 - protein, 52 aa, Artificial Sequence
  human wild-type beta defensin 126 (DEFB-126, DEFB126, DEFB-26, DEFB26, hBD-26) C-terminal sequence
VPADRRANYPVFCVQTKTTRISTVTATTATTTLMMTTASMSSMAPTPVSPTG

SEQ ID NO: 23 - protein, 78 aa, Artificial Sequence
  human variant beta defensin 126 (DEFB-126) with deletion polymorphism frame shift C-terminal sequence
VPADRRANYPVFCVQTKTTRISTVTATTATTTLMMTTASMSSMAPTRFSHWLNIPASVSCSRIPDSLKQRGLKKKKKK

SEQ ID NO: 24 - DNA, 20 bp, Artificial Sequence
  synthetic wild-type human DEFB-126 nucleotide sequence spanning region encompassing DEFB-126 deletion polymorphism, DEFB-126 subsequence
gctcctacccccgtttctcc

SEQ ID NO: 25 - DNA, 18 bp, Artificial Sequence
  synthetic human variant DEFB-126 nucleotide sequence spanning region encompassing DEFB-126 deletion polymorphism, DEFB-126 subsequence with deletion polymorphism
gctcctacccgtttctcc

SEQ ID NO: 26 - protein, 46 aa, Artificial Sequence
  synthetic wild-type human DEFB-126 polypeptide C-terminus sequence
ANYPVFCVQTKTTRISTVTATTATTTLMMTTASMSSMAPTTVSPTG

SEQ ID NO: 27 - protein, 74 aa, Artificial Sequence
  synthetic human DEFB-126 with deletion polymorphism frame shift C-terminal sequence, DEFB-126 variant polypeptide C-terminal sequence
ANYPVFCVQTKTTRISTVTATTATTTLMMTTASMSSMAPTRFSHWLNIPASVSCSRIPDSLKQRQLKKKKKKKK

SEQ ID NO: 28 - protein, 123 aa, Macaca fascicularis
  crab-eating macaque wild-type beta defensin 126 (DEFB-126, DEFB126, DEFB-26, DEFB26)
MKSLLFTLAVFMLLAQLVSGNLYVKRCLNDIGICKKTCKPEEVRSEHGWVMCGKRKACCVPADKRSAYPSFCVHSKTTKTSTVTARATATTATTATAATPLMISNGLISLMTTMAATPVSPTT

SEQ ID NO: 29 - protein, 134 aa, Macaca fascicularis
  crab-eating macaque wild-type beta defensin 126 (DEFB-126, DEFB126, DEFB-26, DEFB26) amino acids 1-134, cynomolgus macaque DEFB-126 amino acids 1-134
MKSLLFTLAVFMLLAQLVSGNLYVKRCLNDIGICKKTCKPEEVRSEHGWVMCGKRKACCVPADKRSAYPSFCVHSKTTKTSTVTARATATTATTATAATPLMISNGLISLMSYDGRYPCFSHYLNIPASVSCSR

SEQ ID NO: 30 - protein, 121 aa, Artificial Sequence
  human DEFB-126 variant polypeptide with extended C-terminal region residues 1-121
MKSLLFTLAVFMLLAQLVSGNWYVKKCLNDVGICKKKCKPEEMHVKNGWAMCGKQRDCCVPADRRANYPVFCVQTKTTRISTVTATTATTTLMMTTASMSSMAPTRFSHWLNIPASVSCSR

SEQ ID NO: 31 - protein, 134 aa, Artificial Sequence
  synthetic DEFB-126 polypeptide consensus sequence
MKSLLFTLAVFMLLAQLVSGNXYVKXCLNDXGICKKXCKPEEXXXXXGWXMCGKXXXCCVPADXRXXYPXFCVXXKTTXXSTVTAXXXXTTXXXXTAXXXXMXXXXXXXXXXXXXXXXXFSHXLNIPASVSCSR

SEQ ID NO: 32 - protein, 114 aa, Artificial Sequence
  synthetic crab-eating macaque wild-type beta defensin 126 (DEFB-126, DEFB126, DEFB-26, DEFB26) excluding signal sequence, amino acids 21-134, cynomolgus macaque DEFB-126 amino acids 21-134
NLYVKRCLNDIGICKKTCKPEEVRSEHGWVMCGKRKACCVPADKRSAYPSFCVHSKTTKTSTVTARATATTATTATAATPLMISNGLISLMSYDGRYPCFSHYLNIPASVSCSR

SEQ ID NO: 33 - protein, 101 aa, Artificial Sequence
  synthetic human DEFB-126 variant polypeptide with extended C-terminal region excluding signal sequence, residues 21-121
NWYVKKCLNDVGICKKKCKPEEMHVKNGWAMCGKQRDCCVPADRRANYPVFCVQTKTTRISTVTATTATTTLMMTTASMSSMAPTRFSHWLNIPASVSCSR

SEQ ID NO: 34 - protein, 114 aa, Artificial Sequence
  synthetic DEFB-126 polypeptide consensus sequence
NXYVKXCLNDXGICKKXCKPEEXXXXXGWXMCGKXXXCCVPADXRXXYPXFCVXXKTTXXSTVTAXXXXTTXXXXTAXXXXMXXXXXXXXXXXXXXXXXFSHXLNIPASVSCSR

SEQ ID NO: 35 - protein, 6 aa, Artificial Sequence
  synthetic wild-type human DEFB-126 polypeptide C-terminal sequence
TVSPTG

SEQ ID NO: 36 - DNA, 21 bp, Artificial Sequence
  synthetic forward primer DEFB126-154s polynucleotide
aagaatggttgggcaatgtgc

SEQ ID NO: 37 - DNA, 25 bp, Artificial Sequence
  synthetic forward primer DEFB126-199s polynucleotide
gcaaacaaagggactgctgtgttcc

SEQ ID NO: 38 - DNA, 24 bp, Artificial Sequence
  synthetic reverse primer DEFB126-330a polynucleotide
aggagccatcgaagacatcgaagc

SEQ ID NO: 39 - DNA, 23 bp, Artificial Sequence
  synthetic reverse primer DEFB126-409a polynucleotide
ccacaatgctttaatgagtcggg

SEQ ID NO: 40 - DNA, 25 bp, Artificial Sequence
  synthetic forward primer DEFB126-278s polynucleotide
cagcaacaacaactttgatgatgac

SEQ ID NO: 41 - DNA, 24 bp, Artificial Sequence
  synthetic forward primer DEFB126-441s polynucleotide
ccatcagcactatgaccccaggac

SEQ ID NO: 42 - DNA, 25 bp, Artificial Sequence
  synthetic reverse primer DEFB126-546a polynucleotide
gttggcagtatgtttggtggaggtg

SEQ ID NO: 43 - protein, 20 aa, Artificial Sequence
  synthetic crab-eating macaque wild-type beta defensin 126 (DEFB-126, DEFB126, DEFB-26, DEFB26) signal peptide, residues 1-20
MKSLLFTLAVFMLLAQLVSG

SEQ ID NO: 44 - protein, 44 aa, Artificial Sequence
  synthetic crab-eating macaque wild-type beta defensin 126 (DEFB-126, DEFB126, DEFB-26, DEFB26) beta-defensin core domain region, residues 21-64
NLYVKRCLNDIGICKKTCKPEEVRSEHGWVMCGKRKACCVPADK

SEQ ID NO: 45 - protein, 59 aa, Artificial Sequence
  synthetic crab-eating macaque wild-type beta defensin 126 (DEFB-126, DEFB126, DEFB-26, DEFB26) extended carboxyl tail, residues 65-123
RSAYPSFCVHSKTTKTSTVTARATATTATTATAATPLMISNGLISLMTTMAATPVSPTT

SEQ ID NO: 46 - protein, 53 aa, Artificial Sequence
  synthetic human defensin core
NWYVKKCLNDVGICKKKCKPEEMHVKNGWAMCGKQRDCCVPADRRANYPVFCV

SEQ ID NO: 47 - protein, 53 aa, Artificial Sequence
  synthetic cynomolgus macaque defensin core
NLYVKRCLNDIGICKKTCKPEEVRSEHGWVMCGKRKACCVPADKRSAYPSFCV

SEQ ID NO: 48 - protein, 50 aa, Artificial Sequence
  synthetic mouse defensin core
GWYVKKCANTLGNCRKMCRDGEKQTEPATSKCPIGKLCCVLDFKISGHCG

SEQ ID NO: 49 - protein, 53 aa, Artificial Sequence
  synthetic defensin core consensus sequence
NWYVKKCLNDXGICKKXCKPEEXXXEXGWXMCGKXKXCCVPADKRSXYPXFCV

SEQ ID NO: 50 - protein, 119 aa, Artificial Sequence
  Description of Artificial Sequence Synthetic polypeptide
XYPXFCVGHCGGGGQNSDNLVTAGGDEGSSAKASTAAMVGAAAMAGTPTKTSAPAKTSAPAKTSTTTKASNAAKASTTTKASNAAKASAATMAGNTTKVSTAAIASTPAQASTPTKANS